(19) United States
(12) Patent Application Publication
(10) Pub. No.: US 2016/0146755 A1
(43) Pub. Date: May 26, 2016

(54) DEVICES, SYSTEMS, AND METHODS FOR ELECTROPHORESIS

(71) Applicant: Board of Regents, The University of Texas System, Austin, TX (US)

(72) Inventors: Richard M. Crooks, College Station, TX (US); Long Luo, Austin, TX (US); Xiang Li, Austin, TX (US)

(21) Appl. No.: 14/952,008

(22) Filed: Nov. 25, 2015

Related U.S. Application Data
(60) Provisional application No. 62/084,076, filed on Nov. 25, 2014.

(51) Int. Cl. G01N 27/447; G01N 33/487; G01N 27/416

(52) U.S. Cl. CPC G01N 27/44756; G01N 27/4166; G01N 33/48707

(57) ABSTRACT

Disclosed herein are devices that can be used to perform electrophoretic separations as well as methods of using thereof. The devices and methods described herein are inexpensive, user friendly, sensitive, portable, robust, efficient, thin, rapid, and use low voltage. As such, the device and methods are well suited for use in numerous applications including point-of-care (POC) diagnostics.

### CROSS-REFERENCE TO RELATED APPLICATIONS

This application claims benefit of U.S. Provisional Application No. 62/084,076, filed Nov. 25, 2014, which is hereby incorporated herein by reference in its entirety.

### STATEMENT REGARDING FEDERALLY SPONSORED RESEARCH

This invention was made with government support under Grant No. CBET1402242 awarded by the National Science Foundation. The government has certain rights in this invention.

### BACKGROUND

There is a significant interest in the development of paper point-of-care (POC) devices that are cheap, user friendly, robust, sensitive, and portable. Such devices pose an effective solution to the existing economic and healthcare accessibility problems in underdeveloped countries, as well as the growing trend in more affluent societies to become better informed in terms of its health. Although commercial paper-based sensors have been around for about 25 years (e.g., pregnancy test and glucose test strips), few paper POC devices have been successfully commercialized. Such failure to produce trustworthy paper POC devices is a combination of many factors, including poor limits of detection (LOD), high non-specific adsorption (NSA), unstable reagents, long analysis time, complex user-technology interface, detection method, and poor sensitivity.

### SUMMARY

Described herein are devices that can be used to perform electrophoretic separations and/or the isotachophoretic concentration of samples. The devices can comprise a plurality of planar segments with each planar segment comprising a fluid permeable region defined by a fluid impermeable boundary. The plurality of planar segments can be stacked (e.g., to form a stack) such that the plurality of planar segments are parallel and aligned. When stacked, the fluid permeable regions of the plurality of planar segments together can form a fluid permeable column within the stack of segments extending from a first end to a second end. The device can further comprise a first electrode in electrical contact with the first end, a second electrode in electrical contact with the second end, or a combination thereof.

In some embodiments, the device can further comprise a first reservoir in fluid contact with the first end, a second reservoir in fluid contact with the second end, or a combination thereof. The device can further comprise a first separator in fluid contact with the first reservoir and the first end, a second separator in fluid contact with the second reservoir and the second end, or a combination thereof. In some embodiments, the first separator can be located between the first reservoir and the first end, the second separator can be located between the second reservoir and the second end, or a combination thereof.

In some embodiments, the device can further comprise a slip layer. The slip layer can comprise a fluid permeable region defined by a fluid impermeable boundary. The slip layer can be translocated from a retracted position to a deployed position, wherein in the retracted position the fluid permeable region of the slip layer is fluidly independent from the fluid permeable column, and wherein in the deployed position, the fluid permeable region of the slip layer is in fluid contact with the fluid permeable column. The slip layer can serve as a loading layer to introduce a sample into the fluid permeable column. The slip layer can also serve as a collection layer on which an analyte can be collected.

In some embodiments, the plurality of segments are joined together in a sheet. When joined in a sheet, the plurality of segments can optionally be coplanar. For example, the plurality of segments can be joined end to end to form an elongate strip. The stack can be formed by folding the sheet so as to align the segments in a stack. In some embodiments, folding the sheet can comprise accordion folding the sheet.

The devices herein can be fabricated from any suitable material or combination of materials. In some embodiments, the devices can be paper based.

In some embodiments, the devices disclosed herein can comprise two or more fluid permeable columns.

Also disclosed herein are methods of use of the devices disclosed herein. In some embodiments, the method can comprise introducing a sample to the fluid permeable column of the device and applying a potential to the fluid permeable column. In some embodiments, the method can comprise electrophoresis (e.g., the device can be configured to electrophoretically localize and/or separate the sample). In some embodiments, the sample can comprise an analyte. In some embodiments, the potential can be 40 volts (V) or less.

In some embodiments, introducing the sample to the fluid permeable column can comprise translocating the slip layer to the deployed position, wherein the sample is initially located in the fluid permeable region of the slip layer.

In some embodiments, the method can further comprise separating the analyte from the sample (e.g., the method can comprise electrophoretically separating the analyte from the sample). In some embodiments, the method can further comprise accumulating the sample, the analyte, or a combination thereof in a section of the fluid permeable column. The section can comprise one or more of the planar segments, the slip layer, or a combination thereof. In some embodiments, the method can further comprise removing the section of the fluid permeable column to isolate the sample, the analyte, or a combination thereof. In some embodiments, the method can further comprise analyzing the sample, analyte, or a combination thereof to determine a property of the sample, the analyte, or a combination thereof.

Also disclosed herein are methods of use of the devices comprising a first fluid permeable column and a second permeable column. For example, the method can comprise a multichannel analysis of one or more sample. In some embodiments, the method can comprise a multi-step analysis, where a sample is loaded into the first fluid permeable column, partially separated such that a first analyte is collected in a section of the first fluid permeable column comprising a slip layer, then translocating the slip layer to introduce the first analyte to the second fluid permeable column and perform another analysis step.

The devices and methods described herein are inexpensive, user friendly, sensitive, portable, robust, efficient, thin (e.g., column is ˜2 mm in length), rapid (completion of analysis in ˜5 min), and use low voltage (e.g., 10-20 V). As such, the device and methods are well suited for use in numerous applications including point-of-care (POC) diagnostics.

### DESCRIPTION OF FIGURES

FIG. 1 displays a schematic view of (A) the planar segment and (B) the device.

FIG. 2 displays a schematic view of a device including the first reservoir, second reservoir, first separator and second separator.

FIG. 3 displays a schematic view of (A) the slip layer, (B) the slip layer in the retracted position within the device, and (C) the slip layer in the deployed position within the device.

FIG. 4 displays a schematic example of the device comprising an accordion folded planar sheet as the stack.

FIG. 5 displays a schematic view of (A) the planar segment with a first fluid permeable region and a second fluid permeable region, and (B) a device with a first fluid permeable column and a second fluid permeable column.

FIG. 6 displays a schematic view of a multi-channel device.

FIG. 7 displays a schematic view of a device used for a multi-step analysis.

FIG. 8 displays the oPAD-Ep device used for separation of proteins in bovine serum.

FIG. 9 displays the designs of the oPAD-Ep components: (a) origami paper, (b) slip layer, and (c) plastic buffer reservoirs. (d) Photographs of the oPAD-Ep.

FIG. 10 displays the fluorescence spectra of BODIPY²⁻, MPTS³⁻, PTS⁴⁻, Ru(bpy)₃²⁺, and Rhodamine 6G.

FIG. 11 displays (a) fluorescence micrographs of a 23-layer oPAD-Ep after Ep of BODIPY²⁻ for run times ranging from 0 min to 6.0 min at 10.0 V and, in the bottom frame, after 6.0 min with no applied voltage. Fluorescence from just the first 20 layers is shown because the last three layers are at the background level. A 16-level color scale was used to differentiate the fluorescence intensities. BODIPY²⁻ (0.50 μL, 1.0 mM) was initially spotted on the slip layer, which is located at Position 3. The white arrow in the fourth micrograph indicates the direction of BODIPY²⁻ migration. (b) Integrated relative fluorescence unit (RFU) distributions, extracted from (a), as a function of Ep run time. The black line is a Gaussian fit to the histograms. (c) Peak positions derived from the Gaussian fittings in (b) as a function of time. The error bars represent the standard deviation of at least three independent tests at each time.

FIG. 12 displays the (a) wax pattern of the device used in the control experiments: black, wax; white, paper. (b) Cross-sectional illustration of the device: orange, Cu cathode; blue, self-laminating pouch; grey, paper channel. Photographs of the device in (c) room light and (d) under a UV lamp after the application of the indicated voltages for 5.0 min.

FIG. 13 displays the correlation of the squared standard deviation (σ²) with time (in minutes) for Ep. The data were obtained from the Gaussian fits shown in FIG. 11b.

FIG. 14 displays the distributions of the integrated RFU for Rhodamine B in a 23-layer oPAD-Ep after Ep at an applied voltage of 10.0 V for run times ranging from 0 to 6.0 min and for 6.0 min with no applied voltage (top frame). Rhodamine B (0.50 μL, 0.10 mM) was initially loaded onto the slip layer at Position 21, and the direction of applied electric field was from Position 23 toward Position 1. The running buffer was 0.20 M Tris-HCl (pH=8.0).

FIG. 15 displays Ep of PTS⁴⁻, MPTS³⁻ and Ru(bpy)₃²⁺ using 23-layer oPAD-Eps and an applied voltage of 10.0 V for different Ep run times ranging from 0 to 6.0 min. PTS⁴⁻ or MPTS³⁻ (0.50 μL, 5.0 mM) were initially pipetted onto the slip layer which is located at Position 3. Due to the positive charge of Ru(bpy)₃²⁺, its slip layer is at Position 21. (a), (c), and (e) show the integrated RFU distributions of these molecules in oPAD-Eps as a function of Ep run time. The black curves are Gaussian fits of the histograms. (b), (d), and (f) are the peak positions derived from the Gaussian fits in (a), (c), and (e) as a function of time.

FIG. 16 displays the histograms of the integrated RFU as a function of position and time for Ep of Rhodamine 6G in a 23-layer oPAD-Ep. Rhodamine 6G (0.50 μL, 0.10 mM) was initially loaded at Position 21 and the direction of the applied electric field (10.0 V) is from Position 23 to 1.

FIG. 17 displays (a) the separation of a mixture of 1.5 mM MPTS³⁻ and 1.5 mM Ru(bpy)₃²⁺ using a 23-layer oPAD-Ep and an applied voltage of 10.0 V. A 0.50 μL aliquot of this mixture was initially spotted on the slip layer located at Position 11. The two arrows in the fourth histogram indicate the directions of MPTS³⁻ and Ru(bpy)₃²⁺ migration. The blue and red histograms correspond to the distributions of Ru(bpy)₃²⁺ and MPTS³⁻, respectively. (b) Fluorescence micrographs of MPTS³⁻ and Ru(bpy)₃²⁺ in the same oPAD-Ep as in (a) after a 3.0 min separation using an applied voltage of 10.0 V. (c) Similar experiment as in (a), but for a mixture of 1.5 mM PTS⁴⁻ and 0.50 mM BODIPY²⁻. A 0.50 μL aliquot of this mixture was initially added to the slip layer at Position 3. (d) Fluorescence micrographs of PTS⁴⁻ and BODIPY²⁻ in the same oPAD-Ep as in (c) after 5.0 min Ep at an applied voltage of 10.0 V.

FIG. 18 displays the Ep of bovine serum albumin (BSA) and bovine IgG at 10.0 V using 11-layer oPAD-Eps. Both BSA and IgG were stained with epicocconone to produce fluorescent conjugates. (a) Fluorescence micrographs of the oPAD-Ep after Ep of BSA for 5.0 min at 10.0 V and, in the second and third frames, after 5.0 min and at 0 min with no applied voltage. BSA (0.50 μL, 5.0 g/dL prepared in 0.1×PBS (ionic strength: 16.3 mM, pH=7.4)) was initially loaded on the slip layer which is at Position 3. The same procedure was used for a 1.0 g/dL bovine IgG solution, and the fluorescence micrographs are shown in (c). (b) and (d) are the corresponding histograms of integrated RFU extracted from (a) and (c), respectively.

FIG. 19 displays the separation of calf serum in 11-layer oPAD-Eps at an applied voltage of 10.0 V. (a) Fluorescence micrographs obtained after Ep of calf serum at 10.0 V for 5.0 min. A 0.50 μL aliquot of serum was initially spotted onto the slip layer at Position 3. (b) and (c) are fluorescence micrographs of oPAD-Eps used for single-component control experiments: 5.0 g/dL BSA and 1.0 g/dL bovine IgG. Separation conditions were the same for all the data in this figure.

FIG. 20 displays the integrated RFU of stained BSA in 3.5-mm-diameter paper zones as a function of BSA concentration.

FIG. 21 illustrates the diagnosis of immunoglobulin deficiency and hepatic cirrhosis based on the results of serum protein separation.

FIG. 22 displays a schematic of the isotachophoresis method.

FIG. 23 displays a schematic of an oPAD used for performing isotachophoresis.

FIG. 24 displays the results of performing isotachophoretic pre-concentration of 23mer DNA with a fluorescent tag on an oPAD device at 20 V for 8 min.

FIG. 25 displays a schematic of a DNA detection assay for diagnostic applications using isotachophoresis on an oPAD device.

FIG. 26 shows a schematic of the origami paper analytical device for isotachophoresis (oPAD-ITP).

FIG. 27 shows a schematic cross-section of the assembled oPAD-ITP.

FIG. 28 shows a schematic of DNA focusing on the oPAD-ITP.

FIG. 29 shows a CorelDRAW drawing of the origami paper device. The white parts represent unmodified paper and the gray areas are impregnated with wax.

FIG. 30 displays a drawing (Autodesk 123D Design) of the 3D-printed reservoirs for the oPAD-ITP.

FIG. 31 displays a photograph of an actual oPAD-ITP. The origami paper is sandwiched between the two green reservoirs using four screws at the corners. Electrodes are inserted into the top holes on the reservoirs.

FIG. 32 displays a photograph of the gel electrophoresis arrangement. After isotachophoresis, each paper layer of the oPAD-ITP was cut off, dried, and inserted into a 1.3% agarose gel containing 10 μg/mL EtBr for analysis by gel electrophoresis. This photograph shows the individual folds inserted into the gel. A fluorescence scanner was used to image the gel after gel electrophoresis.

FIG. 33 displays time-resolved fluorescence micrographs of ssDNA during isotachophoresis focusing of ssDNA using an 11-layer oPAD-ITP at 18 V. The trailing electrolyte and leading electrolyte were 2.0 mM tris-taurine (pH 8.7) and 1.0 M tris-HCl (pH 7.3), respectively, and the initial trailing electrolyte/leading electrolyte boundary was between Layers 2 and 3. The initial ssDNA concentration in the trailing electrolyte solution was 40.0 nM.

FIG. 34 displays the ssDNA calibration curve. For each data point, an 11-layer origami paper (same as the oPAD-ITP) was prepared, and 15.0 μL ssDNA solution having the indicated concentrations was added to the inlet and allowed to wet all 11 layers. Excess liquid was removed, the paper was dried in the dark, and then the fluorescence image of each layer was obtained. The average relative fluorescence unit (RFU) intensity of all layers is plotted as a function of the ssDNA concentration.

FIG. 35 displays the distributions of ssDNA in the oPAD-ITP as a function of time. A 1.0 mL solution containing 40.0 nM ssDNA and trailing electrolyte (TE) buffer was added to the trailing electrolyte reservoir, and 1.0 mL of the leading electrolyte (LE) buffer was added to the leading electrolyte reservoir. After applying a voltage of 18 V for different lengths of time, the oPAD-ITP was unassembled, the individual paper layers were cut out, dried in the dark, and then imaged using fluorescence microscopy. The integrated fluorescence intensities are plotted here.

FIG. 36 displays the plot of the ssDNA peak concentration as a function of time during isotachophoresis focusing of ssDNA using an 11-layer oPAD-ITP. The peak concentrations were calculated from the images in FIG. 33. The error bars represent the standard deviation for three independent replicates.

FIG. 37 displays the plot of peak position (in terms of layer number) as a function of time during isotachophoresis focusing of ssDNA using an 11-layer oPAD-ITP. The peak positions were obtained by Gaussian fitting of the ssDNA distributions shown in FIG. 35. The error bars represent the standard deviation for three independent replicates.

FIG. 38 displays the plot of collection efficiency (C %) as a function of time for ssDNA using an 11-layer oPAD-ITP. The collection efficiency was calculated by comparing the amount of ssDNA injected into the device with the sum of the amount of ssDNA present on each paper layer following isotachophoresis. The error bars represent the standard deviation for three independent replicates.

FIG. 39 displays a current vs time curve for a typical isotachophoresis experiment. In this case, the isotachophoresis voltage (18 V) was applied using a CHI 650C potentiostat (CH Instrument, Austin, Tex.) so that the current could be recorded. Five replicates are shown.

FIG. 40 displays the plot of extraction efficiency (η) as a function of time for ssDNA using an 11-layer oPAD-ITP. The definition of η is given in equation 5. The error bars represent the standard deviation for three independent replicates.

FIG. 41 displays the distribution of ssDNA as a function of position (paper layer number) and initial ssDNA concentration after 4.0 min of isotachophoresis at 18 V. Each data point was calculated by integrating the relative fluorescence unit (RFU) of the fluorescence image of each paper layer. The error bars represent the standard deviation for three independent replicates.

FIG. 42 displays the enrichment factor (EF) and collection efficiency (C %) as a function of the initial ssDNA concentration. The ssDNA concentration was calculated by comparing integrated relative fluorescence unit (RFU) value with the calibration curve shown in FIG. 34. The error bars represent the standard deviation for three independent replicates.

FIG. 43 displays the electroosmotic flow control experiment. The distribution of ssDNA when there was 3.0% polyvinylpyrrolidone (PVP) initially present in the leading electrolyte (LE) to suppress electroosmotic flow (EOF) and the same experiment without PVP are shown. Both isotachophoresis experiments were run at 18 V for 4.0 min. The results show that electroosmotic flow did not significantly affect the value of the enrichment factor (EF) and collection efficiency (C %) in the oPAD-ITP.

FIG. 44 shows the distributions of the nonfocusing fluorescent tracer, Ru(bpy)₃²⁺, in an oPAD-ITP before application of the voltage and at t=4 min. Initially, the leading electrolyte solution contained 30.0 μM Ru(bpy)₃²⁺. Each data point represents the integrated relative fluorescence unit (RFU) value of the fluorescence image of each paper layer. The dash lines represent the guidelines for the nonfocusing fluorescent tracer distributions, which corresponding to electric field strength in the channel. Pt wire electrodes were used. For all experiments the applied voltage was 18 V.

FIG. 45 shows the distribution of the nonfocusing fluorescent tracer, Ru(bpy)₃²⁺, in an oPAD-ITP before application of the voltage and at t=4 min. For this experiment both reservoirs were filled with leading electrolyte solution, but the nonfocusing fluorescent tracer was only present in the anodic reservoir adjacent to Layer 11. Each data point represents the integrated relative fluorescence unit (RFU) value of the fluorescence image of each paper layer. The dash lines represent the guidelines for the nonfocusing fluorescent tracer distributions, which corresponding to electric field strength in the channel. To avoid oxidation of Cl⁻ at the anode, Ag/AgCl wire electrodes were used. For all experiments the applied voltage was 18 V.

FIG. 46 shows the isotachophoresis focusing of a 100 bp dsDNA ladder. Initially, 1.0 μL of a 500 μg/mL solution of the 100 bp dsDNA ladder was dissolved in 1.0 mL of the trailing electrolyte solution (final dsDNA concentration: 0.5 μg/mL). Following 10 min of isotachophoresis of this solution at 18 V, each fold of the 11-layer oPAD-ITP was cut from the device, and gel electrophoresis was used to elute the dsDNA in that layer (FIG. 32). The left panel is a fluorescence micrograph of the gel after electrophoresis and staining with 10 μg/mL EtBr. The numbers under the lanes of the gel correspond to the Layer numbers of the oPAD-ITP. The right panel is a control experiment showing the result of gel electrophoresis of a paper fold onto which 1.0 μL of a 500 μg/mL dsDNA ladder solution was dispensed (no isotachophoresis). The gel electrophoresis conditions for all paper folds were: 1.3% agarose gel, 100 V, and 50 min.

FIG. 47 shows the fluorescence line profiles of the stained gels in each lane of the left panel in FIG. 46 (solid lines). The integral of the profiles is represented by the solid bars.

FIG. 48 shows the total dsDNA placed in the reservoir prior to isotachophoresis (hollow bars) and the total collected dsDNA (solid bars) on the oPAD-ITP. The calculated collection efficiency (C %) values for each dsDNA length are shown in the right-most column.

FIG. 49 shows the enrichment factor (EF) analysis of the isotachophoresis of a dsDNA ladder. Same experiment setup and buffer conditions were used as for the ssDNA focusing experiments. The dsDNA ladder was loaded into trailing electrolyte (TE) buffer. After isotachophoresis at 18 V for 10 min, the oPAD-ITP was unfolded and each paper layer was cut off and inserted in gel as FIG. 32 for gel electrophoresis. The gel electrophoresis image of each DNA band in each lane was analyzed using ImageJ software. The enrichment factor plotted here was calculated as the area of the solid bars in FIG. 47 divided by the area of corresponding hollow bars in FIG. 48.

FIG. 50 schematically illustrates methods for electrochemically detecting an analyte in a single layer of the devices described herein.

FIG. 51 illustrates a method for the electrochemical detection of silver nanoparticles (AgNPs) in a single layer of the devices described herein. AgNPs are an example of an electrochemical label that can be detected to identify and/or quantify an analyte of interest.

FIG. 52 is a linear stripping voltammogram obtained as a result of the method schematically illustrated in FIG. 52. As demonstrated by FIG. 52, the AgNPs present in the paper layer could be effectively detected and quantified using linear stripping voltammetry.

### DETAILED DESCRIPTION

The methods and devices described herein may be understood more readily by reference to the following detailed description of specific aspects of the disclosed subject matter, figures and the examples included therein.

Before the present devices and methods are disclosed and described, it is to be understood that the aspects described below are not intended to be scope by the specific devices and methods described herein, which are intended as illustrations. Various modifications of the devices and methods in addition to those shown and described herein are intended to fall within the scope of that described herein. Further, while only certain representative devices and method steps disclosed herein are specifically described, other combinations of the devices and method steps also are intended to fall within the scope of that described herein, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

Throughout the specification, the terms “planar” and “parallel” are used to describe segments and the relative arrangement of segments. It will be understood that such terms allow for some variation (e.g., segments need not be absolutely planar or parallel but merely substantially planar or parallel) provided that device function is not compromised (e.g., provided that the fluid impermeable boundary that defines the fluid permeable region and by extension to the electrophoretic column remains sufficiently continuous to allow for a device to function).

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

Devices

Disclosed herein are devices 100 that can comprise a plurality of planar segments 104. Referring now to FIG. 1A, in some embodiments, each planar segment 104 can comprise: a top surface 132; a bottom surface 134; and a fluid permeable region 116 defined by a fluid impermeable boundary 118. In some embodiments, the fluid permeable region 116 can extend through the planar segment 104 from the top surface 132 to the bottom surface 134 so as to form a fluid permeable pathway 136 extending through the planar segment 104 from the top surface 132 to the bottom surface 134.

Referring now to FIG. 1B, in some embodiments, the plurality of planar segments 104 can be stacked (e.g., to form a stack 102) such that the plurality of planar segments 104 are parallel. In other words, in some embodiments the device 100 can comprise a stack 102 comprising a plurality of parallel and aligned planar segments 104. The stack 102 can comprise any number of planar segments 104. In some embodiments, the stack 102 can comprise 3 or more planar segments 104 (e.g., 4 or more planar segments 104, 5 or more planar segments 104, 6 or more planar segments 104, 7 or more planar segments 104, 8 or more planar segments 104, 9 or more planar segments 104, 10 or more planar segments 104, 12 or more planar segments 104, 14 or more planar segments 104, 16 or more planar segments 104, 18 or more planar segments 104, 20 or more planar segments 104, 22 or more planar segments 104, 24 or more planar segments 104, 26 or more planar segments 104, 28 or more planar segments 104, 30 or more planar segments 104, 32 or more planar segments 104, 34 or more planar segments 104, 36 or more planar segments 104, 38 or more planar segments 104, or 40 or more planar segments 104). In some embodiments, the stack 102 can comprise 5 layers, such as layers 1, 2, 3, 4, 5 as shown in FIG. 1B, wherein the numbers 1, 2, 3, 4, 5 are merely illustrative.

In some embodiments, the plurality of planar segments 104 can be stacked such that the bottom surface 134 of a first planar segment 104 and the top surface 132 of a second segment 104 are in intimate contact at a juncture 140.

The fluid permeable regions 116 can form a fluid permeable column 106 within the stacked plurality of segments 104 (e.g., within the stack 102) extending from a first end 108 to a second end 110, wherein the first end 108 can comprise the fluid permeable region 116 at the top surface 132 of the first planar segment 104, and wherein the second end 110 can comprise the fluid permeable region 116 at the bottom surface 134 of the last planar segment 104.

In some embodiments, the fluid permeable column 106 can be 10 mm or less in length (e.g., 9.5 mm or less, 9 mm or less, 8.5 mm or less, 8 mm or less, 7.5 mm or less, 7 mm or less, 6.5 mm or less, 6 mm or less, 5.5 mm or less, 5 mm or less, 4.5 mm or less, 4 mm or less, 3.5 mm or less, 3 mm or less, 2.5 mm or less, 2.4 mm or less, 2.3 mm or less, 2.2 mm or less, 2.1 mm or less, 2 mm or less, 1.9 mm or less, 1.8 mm or less, 1.7 mm or less, 1.6 mm or less, 1.5 mm or less, 1.4 mm or less, 1.3 mm or less, 1.2 mm or less, 1.1 mm or less, 1 mm or less, 0.9 mm or less, 0.8 mm or less, 0.7 mm or less, 0.6 mm or less, or 0.5 mm or less), wherein the length is the distance from the first end 108 to the second end 110.

The device 100 can further comprise a first electrode 112 in electrical contact with the first end 108, a second electrode 114 in electrical contact with the second end 110, or a combination thereof.

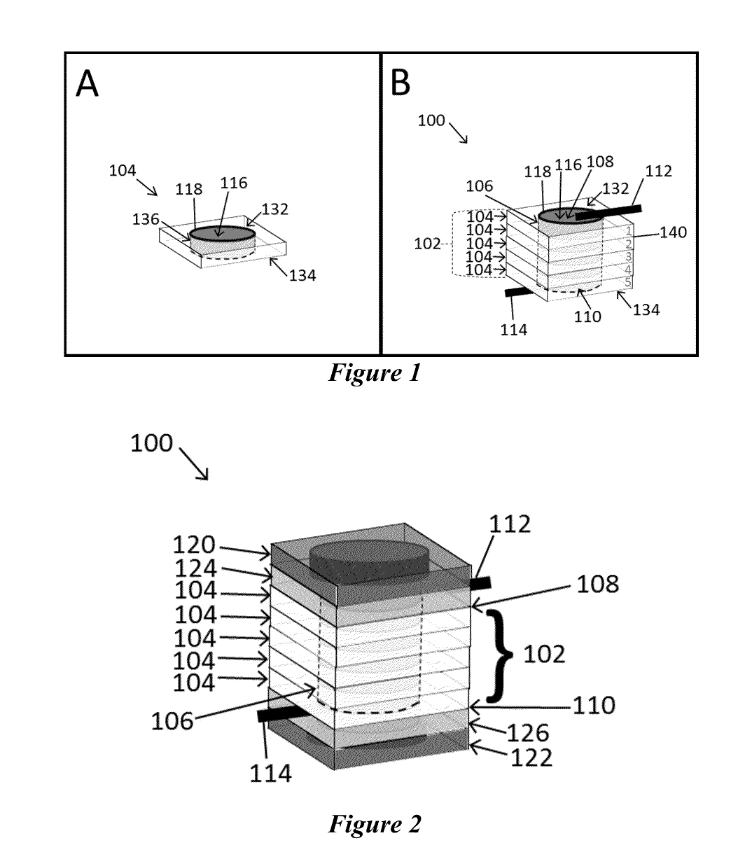

Referring now to FIG. 2, device 100 can further comprise a first reservoir 120 in fluid contact with the first end 108, a second reservoir 122 in fluid contact with the second end 110, or a combination thereof. The first reservoir, the second reservoir, or a combination thereof can comprise a housing, a volume of fluid, or a combination thereof. The fluid can be any fluid consistent with the devices and methods described herein. For example, the fluid can comprise a solvent, an aqueous fluid (e.g., water, buffer solution, etc.), an organic fluid (e.g., toluene, dimethyl-formamide, etc.), and the like. The housing can comprise any material consistent with the methods and devices described herein. For example, the housing can comprise a polymer, metal, glass, wood, or paper. In some embodiments, the housing can comprise an inert, non-absorbent polymer such as a polyether block amide (e.g., PEBAX®, commercially available from Arkema, Colombes, France), a polyacrylate, a polymethacrylate (e.g., poly(methyl methacrylate)), a polyimide, polyurethane, polyamide (e.g., Nylon 6,6), polyvinylchloride, polyester, (HYTREL®, commercially available from DuPont, Wilmington, Del.), polyethylene (PE), polyether ether ketone (PEEK), fluoropolymers such as polytetrafluoroethylene (PTFE), perfluoroalkoxy, fluorinated ethylene propylene, or a blend or copolymer thereof. Silastic materials and silicon based polymers can also be used.

The device 100 can further comprise a first separator 124 in fluid contact with the first reservoir 120 and the first end 108, a second separator 126 in fluid contact with the second reservoir 122 and the second end 110, or a combination thereof. The first separator 124, the second separator 126, or a combination thereof can, for example, separate the fluid permeable region 116 of the device 100 from the volume of solution in the first reservoir 120, the second reservoir 122, or a combination thereof, to prevent the fluid permeable region 116 from being damaged by long-term exposure to solution, undesirable pH changes, the effects of pressure-driven flow, and the like. In some examples, the first separator 124, the second separator 126, or a combination thereof can comprise a housing, a separation material, or a combination thereof. In some examples, the separation material can comprise a hydrogel (e.g., an agar gel).

In some embodiments, the first separator 124 can be located between the first reservoir 120 and the first end 108, the second separator 126 can be located between the second reservoir 122 and the second end 110, or a combination thereof.

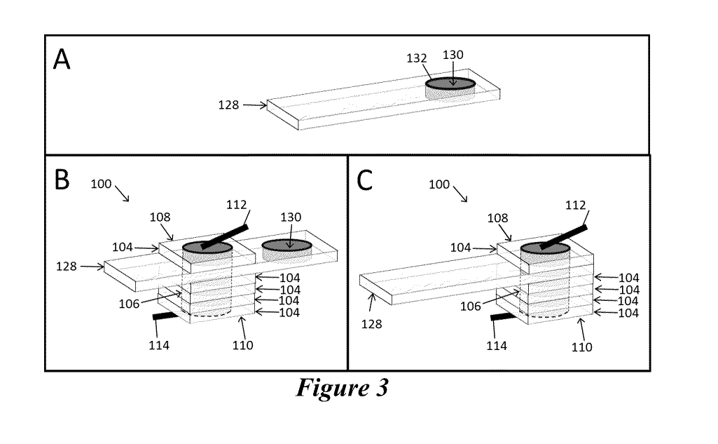

Referring now to FIG. 3, the device 100 can optionally further comprise a slip layer 128. The slip layer 128 can comprise a fluid permeable region 130 defined by a fluid impermeable boundary 132. The slip layer 128 can be translocated from a retracted position (FIG. 3B) to a deployed position (FIG. 3C), wherein in the retracted position (FIG. 3B) the fluid permeable region 130 of the slip layer 128 is fluidly independent from the fluid permeable column 106, and wherein in the deployed position (FIG. 3C), the fluid permeable region 130 of the slip layer 128 is in fluid contact with the fluid permeable column 106.

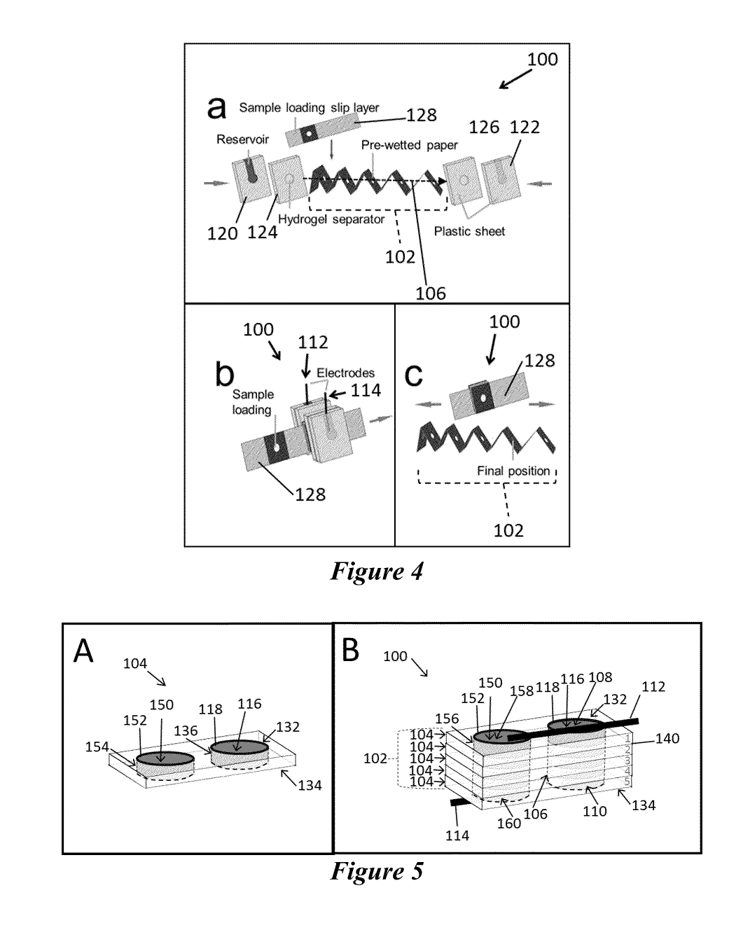

In some embodiments, the plurality of segments 104 can be independent (i.e., non-attached) planar segments that can be stacked to form the devices described herein. In other embodiments, the plurality of segments 104 can be joined together in a sheet. When joined in a sheet, the plurality of segments can optionally be coplanar. In some embodiments, the stack can be formed by folding the sheet, for example as shown in FIG. 4, into an appropriate alignment so as to form the device. The sheet can be folded in any manner consistent with the description of the devices herein, specifically, the sheet can be folded in any manner that aligns the fluid permeable regions 116 to form the fluid permeable column 106. Examples of folding can include zig zag folding, spiral folding, C folding, double parallel folding, gatefolding, double gate folding, French folding, cross folding, and accordion folding. In some embodiments, folding the sheet can comprise accordion folding the sheet.

The devices herein can be fabricated from any suitable material or combination of materials. In some embodiments, the devices 100 can be paper based, meaning that the fluid permeable regions 116 can be formed from a porous, cellulosic substrate such as paper through which fluid flows by wicking. In some cases, the planar segments can be formed from a porous, cellulosic substrate such as paper through which fluid flows by wicking. The dimensions of the permeable regions 116 within the planar segments 104 are defined by a fluid impermeable boundary 118 that substantially permeates the thickness of the planar segment 104, so as to form a boundary that directs fluid flow along the fluid permeable column 106.

The fluid impermeable boundary 118 that defines the fluid permeable region 116 can be formed within a layer of a porous, cellulosic substrate (e.g., within the planar segment 104) using any suitable method known in the art. For example, the fluid impermeable boundary 118 can be formed by wax printing. In these methods, an inkjet printer is used to pattern a wax material on the porous, cellulosic substrate. Many types of wax-based solid ink are commercially available and are useful in such methods as the ink provides a visual indication of the location of the fluid impermeable boundary 118. However, it should be understood, that the wax material used to form the fluid impermeable boundary 118 does not require an ink to be functional. Examples of wax materials that maybe used include polyethylene waxes, hydrocarbon amide waxes or ester waxes. Once the wax is patterned, the porous, cellulosic substrate is heated (e.g., by placing the substrate on a hot plate with the wax side up at a temperature of 120° C.) and cooled to room temperature. This allows the wax material to substantially permeate the thickness of the porous, cellulosic substrate, so as to form a fluid impermeable boundary 118 that defines the dimensions of the fluid permeable region 116.

In some embodiments, the device can be a paper-based device formed from a porous, cellulosic substrate that is flexible. For certain applications, it is preferable that the cellulosic substrate can be folded, creased, or otherwise mechanically shaped to impart structure and function to the paper-based device formed from the cellulosic substrate. Examples of suitable porous, cellulosic substrates for the fabrication of paper-based devices include cellulose; derivatives of cellulose such as nitrocellulose or cellulose acetate; paper (e.g., filter paper, chromatography paper); woven cellulosic materials; and non-woven cellulosic materials.

In some embodiment, the porous, cellulosic substrate is paper. Paper is inexpensive, widely available, readily patterned, thin, lightweight, and can be disposed of with minimal environmental impact. Furthermore, a variety of grades of paper are available, permitting the selection of a paper substrate with the weight (i.e., grammage), thickness and/or rigidity and surface characteristics (i.e., porosity, hydrophobicity, and/or roughness), desired for the fabrication of a particular paper-based device. Suitable papers include, but are not limited to, chromatography paper, card stock, filter paper, vellum paper, printing paper, wrapping paper, ledger paper, bank paper, bond paper, blotting paper, drawing paper, fish paper, tissue paper, paper towel, wax paper, and photography paper.

In some embodiments, the devices described herein can be affixed to or secured within a polymer, metal, glass, wood, or paper housing to facilitate handling and use of the device. In some embodiments, the devices described herein are affixed to or secured within an inert, non-absorbent polymer such as a polyether block amide (e.g., PEBAX®, commercially available from Arkema, Colombes, France), a polyacrylate, a polymethacrylate (e.g., poly(methyl methacrylate)), a polyimide, polyurethane, polyamide (e.g., Nylon 6,6), polyvinylchloride, polyester, (HYTREL®, commercially available from DuPont, Wilmington, Del.), polyethylene (PE), polyether ether ketone (PEEK), fluoropolymers such as polytetrafluoroethylene (PTFE), perfluoroalkoxy, fluorinated ethylene propylene, or a blend or copolymer thereof. Silastic materials and silicon based polymers can also be used.

The devices described herein can be coupled to a power supply and optionally to one or more additional suitable features including, but not limited to, a voltmeter, an ammeter, a multimeter, an ohmmeter, a signal generator, a pulse generator, an oscilloscope, a frequency counter, a potentiostat, or a capacitance meter. The devices described herein can also be coupled to a computing device that performs arithmetic and logic operations necessary to analyze the samples from the device (e.g., to determine analyte concentration, etc.).

Also disclosed herein is a device 100 comprising: a plurality of planar segments 104, each planar segment 104 comprising a fluid permeable region 116 defined by a fluid impermeable boundary 118, wherein when the plurality of planar segments 104 are stacked such that the fluid permeable region 116 of each planar segment 104 is aligned, the fluid permeable region 116 of each planar segment collectively forms a fluid permeable column 106 traversing the stacked plurality of planar segments 104 from a first end 108 to a second end 110.

Also disclosed herein is a device 100 comprising: a stack 102 formed from a plurality of parallel segments 104; a fluid permeable column 106 traversing the stack 102 from a first end 108 to a second end 110; a first electrode 112 in electrical contact with the first end 108; and a second electrode 114 in electrical contact with the second end 110; wherein each parallel segment 104 comprises a fluid permeable region 116 defined by a fluid impermeable boundary 118; and wherein stacking of the plurality of segments 104 aligns the fluid permeable region 116 within each of the plurality of parallel segments 104 to form the fluid permeable column 106.

In some embodiments, the devices disclosed herein can comprise two or more fluid permeable columns. Referring now to FIG. 5A, devices 100 can comprise a plurality of planar segments 104, each planar segment 104 comprising: a top surface 132; a bottom surface 134; a first fluid permeable region 116 defined by a first fluid impermeable boundary 118 extending through the planar segment from the top surface 132 to the bottom surface 134 so as to form a first fluid permeable pathway 136 extending through the planar segment 104 from the top surface 132 to the bottom surface 134; and a second fluid permeable region 150 defined by a second fluid impermeable boundary 152 extending through the planar segment 104 from the top surface 132 to the bottom surface 134 so as to form a second fluid permeable pathway 154 extending through the planar segment 154 from the top surface 132 to the bottom surface 134.

Referring now to FIG. 5B, the plurality of planar segments 104 can be stacked to form a stack 102 such that the plurality of planar segments 104 are parallel and aligned. In other words, in some embodiments the device 100 can comprise a stack 102 comprising a plurality of parallel segments 104. In some embodiments, the plurality of planar segments 104 can be stacked such that there is an intimate contact juncture 140 between the bottom surface 134 of a first planar segment 104 and the top surface 132 of a second planar segment 104. In some embodiments, the first fluid permeable regions 116 together form a first fluid permeable column 106 within the stacked plurality of segments 104 (e.g., within the stack 102) extending from a first end 108 to a second end 110, wherein the first end 108 comprises the fluid permeable region 116 at the top surface 132 of the first planar segment 104 and the second end 110 comprises the fluid permeable region 116 at the bottom surface 134 of the last planar segment 104. In some embodiments, the second fluid permeable regions 150 together form a second fluid permeable column 156 within the stacked plurality of segments 104 extending from a third end 158 to a fourth end 160, wherein the third end 158 comprises the second fluid permeable region 150 at the top surface 132 of the first segment 104 and the fourth end 160 comprises the second fluid permeable region 150 at the bottom surface 134 of the last segment 104. In some embodiments, the device 100 can further comprise a first electrode 112 in electrical contact with the first end 108, the third end 158, or a combination thereof; a second electrode 114 in electrical contact with the second end 110, the fourth end 160, or a combination thereof; or a combination thereof.

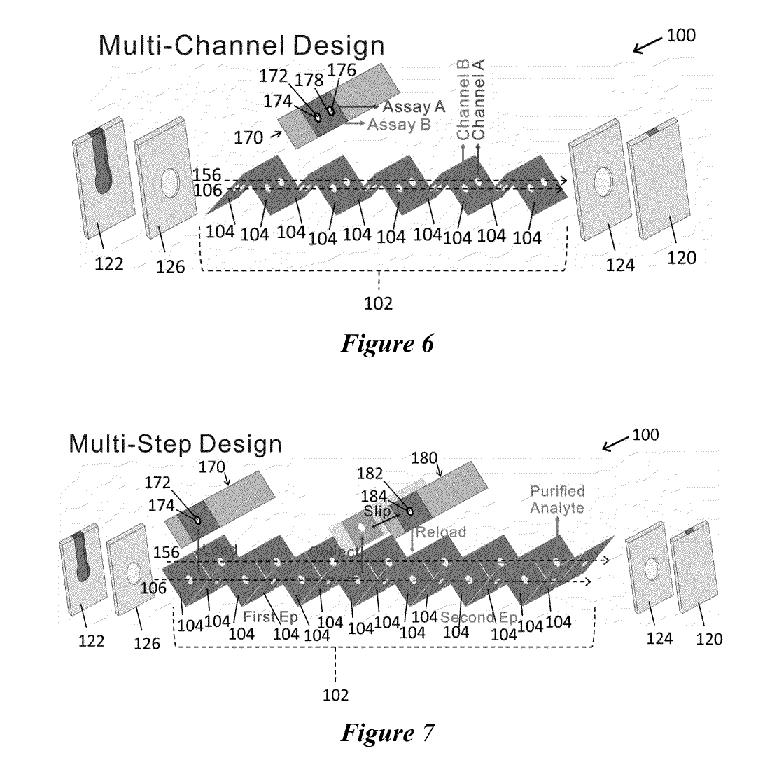

Referring now to FIG. 6, the device 100 can further comprise a first reservoir 120 in fluid contact with the first end 108, the third end 158, or a combination thereof; a second reservoir 122 in fluid contact with the second end 110, the fourth end 160, or a combination thereof; or a combination thereof. In some embodiments, the device 100 can further comprise: a first separator 124 in fluid contact with the first reservoir 120 and the first end 108, the third end 158, or a combination thereof; a second separator 126 in fluid contact with the second reservoir 122 and the second end 110, the fourth end 160, or a combination thereof; or a combination thereof. In some embodiments, a first separator 124 can be located between the first reservoir 120 and the first end 108, the third end 158, or a combination thereof; the second separator 126 can be located between the second reservoir 122 and the second end 110, the fourth end 160, or a combination thereof; or a combination thereof.

The device 100 can optionally further comprise a loading slip layer 170. The loading slip layer 170 can comprise a first fluid permeable region 172 defined by a first fluid impermeable boundary 174, wherein the loading slip layer can be translocated from a retracted position to a deployed position. In the retracted position, the first fluid permeable region 172 of the loading slip layer 170 is fluidly independent from the first fluid permeable column 106 and the second fluid permeable column 156. In the deployed position, the first fluid permeable region 172 of the loading slip layer 170 is in fluid contact with the first fluid permeable column 106, the second fluid permeable column 156, or a combination thereof. In some embodiments, the loading slip layer 170 can further comprise a second fluid permeable region 176 defined by a second fluid impermeable boundary 178, wherein in the retracted position the second fluid permeable region 176 of the loading slip layer 170 is fluidly independent from the first fluid permeable column 106 and the second fluid permeable column 156 and in the deployed position, the second fluid permeable region 176 of the loading slip layer 170 is in fluid contact with the second fluid permeable 156 column.

Referring now to FIG. 7, in some embodiments the device 100 can further comprise a collection slip layer 180, wherein the collection slip layer 180 can comprise a fluid permeable region 182 defined by a fluid impermeable boundary 184. The collection slip layer 180 can be translocated from a first position to a second position. In the first position, the fluid permeable region 182 of the collection slip layer 180 is in fluid contact with the first fluid permeable column 106 and fluidly independent from the second fluid permeable column 156. In the second position, the fluid permeable region 182 of the collection slip layer 180 is in fluid contact with the second fluid permeable column 156 and fluidly independent from the first fluid permeable column 106.

Also disclosed herein is a device 100 comprising: a plurality of planar segments 104, each planar segment 104 comprising a first fluid permeable region 116 defined by a first fluid impermeable boundary 118 and a second fluid permeable region 150 defined by a second fluid impermeable boundary 152; wherein when the plurality of planar segments 104 are stacked such that the first fluid permeable region 116 of each planar segment 104 is aligned and the second fluid permeable region 150 of each planar segment 104 is aligned, the first fluid permeable region 116 of each planar segment 104 collectively forms a first fluid permeable column 116 traversing the stacked plurality of planar segments (e.g., the stack 102) from a first end 108 to a second end 110 and the second fluid permeable region 150 of each planar segment 104 collectively forms a second fluid permeable column 156 traversing the stacked plurality of planar segments (e.g., the stack 102) from a third end 158 to a fourth end 160.

Also disclosed herein is a device 100 comprising: a stack 102 formed from a plurality of parallel segments 104; a first fluid permeable column 106 traversing the stack 102 from a first end 108 to a second end 110; a second fluid permeable column 156 traversing the stack 102 from a third end 158 to a fourth end 160; a first electrode 112 in electrical contact with the first end 108, the third end 158, or a combination thereof; and a second electrode 114 in electrical contact with the second end 110, the fourth end 160, or a combination thereof; wherein each parallel segment 104 comprises a first fluid permeable region 116 defined by a first fluid impermeable boundary 118 and a second fluid permeable region 150 defined by a second fluid impermeable boundary 152; wherein stacking of the plurality of segments 104 aligns the first fluid permeable region 116 within each of the plurality of parallel segments 104 to form the first fluid permeable column 106; and wherein stacking of the plurality of segments 104 aligns the second fluid permeable region 150 within each of the plurality of parallel segments 104 to form the second fluid permeable column 156.

Methods

Also disclosed herein are methods of use of the devices disclosed herein. In some embodiments, the method can comprise introducing a sample to the fluid permeable column of the device and applying a potential to the fluid permeable column. In some embodiments, the method can comprise electrophoresis (e.g., the device can be configured to electrophoretically localize and/or separate the sample). In some embodiments, the method can comprise isotachophoresis (e.g., the device can be configured to separate, localize and/or concentrate the sample).

The sample can comprise any fluid sample of interest. By way of example the fluid sample can be a bodily fluid. “Bodily fluid”, as used herein, refers to a fluid composition obtained from or located within a human or animal subject. Bodily fluids include, but are not limited to, urine, whole blood, blood plasma, serum, tears, semen, saliva, sputum, exhaled breath, nasal secretions, pharyngeal exudates, bronchoalveolar lavage, tracheal aspirations, interstitial fluid, lymph fluid, meningal fluid, amniotic fluid, glandular fluid, feces, perspiration, mucous, vaginal or urethral secretion, cerebrospinal fluid, and transdermal exudate. Bodily fluid also includes experimentally separated fractions of all of the preceding solutions, as well as mixtures containing homogenized solid material, such as feces, tissues, and biopsy samples.

In some embodiments, the sample can comprise an analyte. The analyte can be, for example, a biomarker (i.e., a molecular indicator associated with a particular pathological or physiological state) present in the bodily fluid (e.g., the sample) that can be assayed to identify risk for, diagnosis of, or progression of a pathological or physiological process in a subject. Examples of biomarkers include proteins, hormones, prohormones, lipids, carbohydrates, DNA, RNA, and combinations thereof. The analyte can be, for example, an antibody, peptide (natural, modified, or chemically synthesized), protein (e.g., a glycoprotein, a lipoprotein, or a recombinant protein), polynucleotide (e.g., DNA or RNA, an oligonucleotide, an aptamer, or a DNAzyme), lipid, polysaccharide, small molecule organic compound (e.g., a hormone, a prohormone, a narcotic, or a small molecule pharmaceutical), pathogen (e.g., bacteria, virus, or fungi, or protozoa), or combination thereof

The potential can be any potential consistent with the devices and methods described herein. In some embodiments, the potential applied to the fluid permeable column can be substantially less than the voltage applied in traditional electrophoresis. In some embodiments, the potential can be 40 volts (V) or less (e.g., 38 V or less, 36 V or less, 34 V or less, 32 V or less, 30 V or less, 28 V or less, 26 V or less, 24 V or less, 22 V or less, 20 V or less, 18 V or less, 16 V or less, 14 V or less, 12 V or less, or 10 V or less).

In some embodiments, introducing the sample to the fluid permeable column can comprise translocating the slip layer to the deployed position, wherein the sample is initially located in the fluid permeable region of the slip layer.

In some embodiments, the method can further comprise separating the analyte from the sample (e.g., the method can comprise electrophoretically separating the analyte from the sample).

In some embodiments, the method can further comprise accumulating the sample, the analyte, or a combination thereof in a section of the fluid permeable column. The section can comprise one or more of the parallel segments, the slip layer, or a combination thereof. In some embodiments, the method can further comprise removing the section of the fluid permeable column to isolate the sample, the analyte, or a combination thereof

In some embodiments, the method can further comprise analyzing the sample, analyte, or a combination thereof to determine a property of the sample, the analyte, or a combination thereof. Analyzing the sample, the analyte, or a combination thereof can comprise performing any type of analysis known in the art. Examples of analysis techniques include, but are not limited to, spectroscopic analysis (e.g., atomic absorption spectroscopy, atomic emission spectroscopy, atomic fluorescence spectroscopy, energy dispersive spectroscopy, fluorescence spectroscopy, UV-vis spectroscopy, Raman spectroscopy, X-Ray fluorescence spectroscopy, IR spectroscopy, laser induced breakdown spectroscopy, nuclear magnetic resonance spectroscopy, etc.), chromatographic analysis (e.g., thin layer chromatography, gas chromatography, etc.), colorimetry, voltammetry, potentiometry, calorimetry (e.g., differential scanning calorimetry), flow injection analysis, electron paramagnetic resonance, gas chromatography-mass spectrometry (GC-MS), gas chromatography-IR spectroscopy (GC-IR), mass-spectrometry, transmission electron microscopy, scanning electron microscopy, thermogravimetric analysis, X-ray diffraction, X-ray microscopy, and combinations thereof. In some embodiments, analyzing the sample, analyte, or a combination thereof can comprise spectroscopic analysis (e.g., fluorescence spectroscopy) of the sample, analyte, or a combination thereof. In some embodiments, analyzing the sample, analyte, or a combination thereof can comprise analyzing the sample, analyte, or a combination thereof comprises electrochemical analysis of the sample, analyte, or a combination thereof. In some cases, this can comprise, for example, electrochemical detection of an electrochemical tag or label, such as a metal nanoparticle, conjugated to the analyte.

The property can be any property of interest of the sample, the analyte or a combination thereof. For example, the property can comprise the concentration of the sample and/or the analyte, determining the presence or absence of a particular analyte within the sample, determining the identity of the analyte, determining the number of analytes within the sample, or a combination thereof.

Also disclosed herein are methods of using devices that comprise a first fluid permeable column and a second permeable column. For example, the method can comprise a multichannel analysis of one or more sample, such as that illustrated in FIG. 6. In some embodiments, the method can comprise a multi-step analysis, such as that illustrated in FIG. 7, where a sample is loaded into the first fluid permeable column, partially separated such that a first analyte is collected in a section of the first fluid permeable column comprising a slip layer, then translocating the slip layer to introduce the first analyte to the second fluid permeable column and perform another analysis step.

In some embodiments, the method can comprise introducing a sample to the first fluid permeable column, the second fluid permeable column, or a combination thereof; and applying a potential to the first fluid permeable column, the second fluid permeable column, or a combination thereof. Introducing the sample to the first fluid permeable column, the second fluid permeable column, or a combination thereof can comprise, for example, translocating the loading slip layer to the deployed position, wherein the sample is initially located in the first fluid permeable region of the loading slip layer, the second fluid permeable region of the loading slip layer, or a combination thereof.

In some embodiments, the sample can comprise a first analyte. In some embodiments, the method can further comprise separating the first analyte from the sample. In some embodiments, the method can further comprise accumulating the sample, the first analyte, or a combination thereof in a section of the first fluid permeable column, the second fluid permeable column, or a combination thereof. In some embodiments, the section can comprise one or more of the parallel segments, the loading slip layer, the collection slip layer, or a combination thereof. In some embodiments, the method can further comprise removing the section of the first fluid permeable column, the second fluid permeable column, or a combination thereof to isolate the sample, the first analyte, or a combination thereof. In some embodiments, the method can further comprise analyzing the sample, the first analyte, or a combination thereof to determine a property of the sample, the first analyte, or a combination thereof.

In some embodiments, the section of the first fluid permeable column can comprise the collection slip layer in the first position. In some embodiments, the method can further comprise translocating the collection slip layer to the second position. In some embodiments, the method can further comprise applying a potential to the second fluid permeable column. In some embodiments, the sample the first analyte, or a combination thereof further can comprise a second analyte. In some embodiments, the method can further comprise separating the second analyte from the sample, the first analyte, or a combination thereof. In some embodiments, the method can further comprise accumulating the sample, the first analyte, the second analyte or a combination thereof in a section of the second fluid permeable column. In some embodiments, the method can further comprise removing the section of the second fluid permeable column to isolate the sample, the first analyte, the second analyte, or a combination thereof. In some embodiments, the section of the second fluid permeable column can comprise one or more of the parallel segments, the loading slip layer, the collection slip layer, or a combination thereof. In some embodiments, the method can further comprise analyzing the sample, the first analyte, the second analyte, or a combination thereof to determine a property of the sample, the first analyte, the second analyte, or a combination thereof.

In some embodiments the method can comprise introducing a first sample to the first fluid permeable column, introducing a second sample to the second fluid permeable column, and applying a potential to the first fluid permeable column and the second fluid permeable column (e.g., FIG. 6). The first sample can be the same or different than the second sample. Introducing the first sample to the first fluid permeable column and the second sample to the second fluid permeable column can comprise, for example, translocating the loading slip layer to the deployed position, wherein the first sample is initially located in the first fluid permeable region of the loading slip layer and the second sample is initially located in the second fluid permeable region of the loading slip layer, or a combination thereof.

In some embodiments, the first sample can comprise a first analyte. In some embodiments, the second sample can comprise a second analyte. In some embodiments, the method can further comprise separating the first analyte from the first sample, separating the second analyte from the second sample, or a combination thereof. In some embodiments, the method can further comprise accumulating the first sample, the first analyte, or a combination thereof in a section of the first fluid permeable column; accumulating the second sample, the second analyte or a combination thereof in a section of the second fluid permeable column; or a combination thereof. In some embodiments, the section can comprise one or more of the parallel segments, the loading slip layer, the collection slip layer, or a combination thereof. In some embodiments, the method can further comprise removing the section of the first fluid permeable column to isolate the first sample, the first analyte, or a combination thereof; removing the section of the second fluid permeable column to isolate the second sample, the second analyte, or a combination thereof; or a combination thereof. In some embodiments, the method can further comprise analyzing the first sample, the second sample, the first analyte, the second analyte, or a combination thereof to determine a property of the first sample, the second sample, the first analyte, the second analyte, or a combination thereof.

The devices and methods described herein are inexpensive, user friendly, sensitive, portable, robust, efficient, thin (e.g., column is ˜2 mm in length), rapid (completion of analysis in ˜5 min), and use low voltage (e.g., 10-20 V). As such, the device and methods are well suited for use in numerous sensing applications.

For example, the devices and methods described herein can be used in clinical and healthcare settings to detect and/or quantify biomarkers to identify risk for, diagnosis of, or progression of a pathological or physiological process in a subject. Examples of biomarkers include proteins, hormones, prohormones, lipids, carbohydrates, DNA, RNA, and combinations thereof.

The devices and methods described herein can be used in POC applications to diagnose infections in a patient (e.g., by measuring serum antibody concentrations or detect antigens). For example, the devices and methods described herein can be used to diagnose viral infections (e.g., HIV, hepatitis B, hepatitis C, rotavirus, influenza, polio, measles, yellow fever, rabies, dengue, or West Nile Virus), bacterial infections (e.g., E. coli, C. tetani, cholera, typhoid, diphtheria, tuberculosis, plague, Lyme disease, or H. pylori), and parasitic infections (e.g., toxoplasmosis, Chagas disease, or malaria). The devices and methods described herein can be used to rapidly assesses the immune status of people or animals against selected vaccine-preventable diseases (e.g. anthrax, human papillomavirus (HPV), diphtheria, hepatitis A, hepatitis B, haemophilus influenzae type b (Hib), influenza (flu), Japanese encephalitis (JE), measles, meningococcal, mumps, pertussis, pneumococcal, polio, rabies, rotavirus, rubella, shingles (herpes zoster), smallpox, tetanus, typhoid, tuberculosis (TB), varicella (chickenpox), yellow fever). The devices and methods described herein can be used to rapidly screen donated blood for evidence of viral contamination by HIV, hepatitis C, hepatitis B, and HTLV-1 and -2. The devices and methods described herein can also be used to measure hormone levels. For example, the devices and methods described herein can be used to measure levels of human chorionic gonadotropin (hCG) (as a test for pregnancy), Luteinizing Hormone (LH) (to determine the time of ovulation), or Thyroid Stimulating Hormone (TSH) (to assess thyroid function). The devices and methods described herein can be used to diagnose or monitor diabetes in a patient, for example, by measuring levels of glycosylated hemoglobin, insulin, or combinations thereof. The devices and methods described herein can be used to detect protein modifications (e.g., based on a differential charge between the native and modified protein and/or by utilizing recognition elements specific for either the native or modified protein). The devices and methods described herein can be used to administer personalized medical therapies to a subject (e.g., in a pharmacogenomic assay performed to select a therapy to be administered to a subject).

The devices and methods described herein can also be used in other commercial applications. For example, the devices and methods described herein can be used in the food and beverage industry, for example, in quality control applications or to detect potential food allergens, such as milk, peanuts, walnuts, almonds, and eggs. The devices and methods described herein can be used to detect and/or measure the levels of proteins of interest in foods, cosmetics, nutraceuticals, pharmaceuticals, and other consumer products. The devices and methods described herein can also be used to rapidly and accurately detect narcotics and biothreat agents (e.g., ricin).

### EXAMPLE EMBODIMENTS

Certain example embodiments are provided below.

### Embodiment 1

A device comprising:

a stack formed from a plurality of parallel segments;

a fluid permeable column traversing the stack from a first end to a second end;

a first electrode in electrical contact with the first end; and

a second electrode in electrical contact with the second end;

wherein each segment comprises a fluid permeable region defined by a fluid impermeable boundary; and

wherein stacking of the plurality of segments aligns the fluid permeable region within each of the plurality of parallel segments to form the fluid permeable column.

### Embodiment 2

The device of embodiment 1, further comprising a first reservoir in fluid contact with the first end, a second reservoir in contact with the second end, or a combination thereof.

### Embodiment 3

The device of embodiment 2, further comprising a first separator in fluid contact with the first reservoir and the first end, a second separator in fluid contact with the second reservoir and the second end, or a combination thereof.

### Embodiment 4

The device of embodiment 3, wherein the first separator is located between the first reservoir and the first end, second separator is located between the second reservoir and the second end, or a combination thereof.

### Embodiment 5

The device of any one of embodiments 1-4, further comprising a slip layer, wherein the slip layer comprises:

a fluid permeable region defined by a fluid impermeable boundary;

wherein the slip layer can be translocated from a retracted position to a deployed position;

wherein in the retracted position the fluid permeable region of the slip layer is fluidly independent from the fluid permeable column; and

wherein in the deployed position, the fluid permeable region of the slip layer is in fluid contact with the fluid permeable column.

### Embodiment 6

The device of any one of embodiments 1-5, wherein the plurality of segments are joined together in a sheet, and the stack is formed by folding the sheet.

### Embodiment 7

The device of embodiment 6, wherein folding the sheet comprises accordion folding the sheet.

### Embodiment 8

The device of any one of embodiments 1-7, wherein the plurality of parallel segments comprises at least 3 parallel segments.

### Embodiment 9

The device of any one of embodiments 1-8, wherein the fluid permeable column is 10 mm or less in length.

### Embodiment 10

The device of any one of embodiments 1-9, wherein the device is paper based.

### Embodiment 11

The device of any one of embodiments 1-10, wherein the device further comprises a housing.

### Embodiment 12

A method comprising:

introducing a sample to the fluid permeable column of the device of any one of embodiments 1-11; and

applying a potential to the fluid permeable column.

### Embodiment 13

The method of embodiment 12, wherein the potential is 40 V or less.

### Embodiment 14

The method of any one of embodiments 12-13, wherein the sample comprises an analyte.

### Embodiment 15

The method of embodiment 14, further comprising separating the analyte from the sample.

### Embodiment 16

The method of any one of embodiments 12-15, further comprising accumulating the sample, the analyte, or a combination thereof in a section of the fluid permeable column.

### Embodiment 17

The method of embodiment 18, further comprising removing the section of the fluid permeable column to isolate the sample, the analyte, or a combination thereof.

### Embodiment 18

The method of any one of embodiments 18-19, wherein the section can comprise one or more of the parallel segments, a slip layer, or a combination thereof.

### Embodiment 19

The method of any one of embodiments 12-18, further comprising analyzing the sample, analyte, or a combination thereof to determine a property of the sample, the analyte, or a combination thereof.

### Embodiment 20

The method of any one of embodiments 12-19, wherein introducing the sample to the fluid permeable column comprises translocating the slip layer to the deployed position, wherein the sample is initially located in the fluid permeable region of the slip layer.

### Embodiment 21

A device comprising a plurality of planar segments, each planar segment comprising a fluid permeable region defined by a fluid impermeable boundary,

wherein when the plurality of planar segments are stacked such that the fluid permeable region of each planar segment is aligned, the fluid permeable regions of the plurality of planar segments collectively forms a fluid permeable column traversing the stacked plurality of planar segments from a first end to a second end.

### Embodiment 22

The device of embodiment 21, further comprising a first electrode in electrical contact with the first end, a second electrode in electrical contact with the second end, or a combination thereof.

### Embodiment 23

The device of any one of embodiments 21-22, further comprising a first reservoir in fluid contact with the first end, a second reservoir in fluid contact with the second end, or a combination thereof.

### Embodiment 24

The device of embodiment 23, further comprising a first separator in fluid contact with the first reservoir and the first end, a second separator in fluid contact with the second reservoir and the second end, or a combination thereof.

### Embodiment 25

The device of embodiment 24, wherein the first separator is located between the first reservoir and the first end, the second separator is located between the second reservoir and the second end, or a combination thereof.

### Embodiment 26

The device of any one of embodiments 21-25, further comprising a slip layer, wherein the slip layer comprises a fluid permeable region defined by a fluid impermeable boundary, wherein the slip layer can be translocated from a retracted position to a deployed position, wherein in the retracted position the fluid permeable region of the slip layer is fluidly independent from the fluid permeable column, and wherein in the deployed position, the fluid permeable region of the slip layer is in fluid contact with the fluid permeable column.

### Embodiment 27

The device of any one of embodiments 21-26, wherein the plurality of planar segments are joined together in a sheet, and the plurality of planar segments are stacked by folding the sheet.

### Embodiment 28

The device of embodiment 27, wherein folding the sheet comprises accordion folding the sheet.

### Embodiment 29

The device of any one of embodiments 21-28, wherein the plurality of planar segments comprises at least 3 planar segments.

### Embodiment 30

The device of any one of embodiments 21-29, wherein the fluid permeable column is 10 mm or less in length.

### Embodiment 31

The device of any one of embodiments 21-30, wherein the device is paper based.

### Embodiment 32

The device of any one of embodiments 21-31, wherein the device further comprises a housing.

### Embodiment 33

A method comprising:

introducing a sample to the fluid permeable column of the device of any one of embodiments 21-32; and

applying a potential to the fluid permeable column.

### Embodiment 34

The method of embodiment 33, wherein the potential is 40 V or less.

### Embodiment 35

The method of any one of embodiments 33-34, wherein the sample comprises an analyte.

### Embodiment 36

The method of embodiment 35, further comprising separating the analyte from the sample.

### Embodiment 37

The method of any one of embodiments 33-36, further comprising accumulating the sample, analyte, or a combination thereof in a section of the fluid permeable column.

### Embodiment 38

The method of embodiment 37, further comprising removing the section of the fluid permeable column to isolate the sample, the analyte, or a combination thereof.

### Embodiment 39

The method of any one of embodiments 37-38, wherein the section can comprise one or more of the planar segments, a slip layer, or a combination thereof.

### Embodiment 40

The method of any one of embodiments 33-39, further comprising analyzing the sample, analyte, or a combination thereof to determine a property of the sample, the analyte, or a combination thereof.

### Embodiment 41

The method of any one of embodiments 33-40, wherein introducing the sample to the fluid permeable column comprises translocating the slip layer to the deployed position, wherein the sample is initially located in the fluid permeable region of the slip layer.

### Embodiment 42

A device comprising:

a plurality of planar segments, each planar segment comprising:

a top surface;

a bottom surface; and

a fluid permeable region defined by a fluid impermeable boundary extending through the planar segment from the top surface to the bottom surface so as to form a fluid permeable pathway extending through the planar segment from the top surface to the bottom surface;

wherein when the plurality of planar segments are stacked such that the bottom surface of a first planar segment is in intimate contact with the top surface of a second planar segment, the fluid permeable regions together form a fluid permeable column within the stacked plurality of planar segments extending from a first end to a second end;

wherein the first end comprises the fluid permeable region at the top surface of the first planar segment;

wherein the second end with the fluid permeable region at the bottom surface of the last planar segment;

a first electrode in electrical contact with the first end;

a second electrode in electrical contact with the second end.

### Embodiment 43

The device of embodiment 42, further comprising a first reservoir in fluid contact with the first end, a second reservoir in fluid contact with the second end, or a combination thereof.

### Embodiment 44

The device of embodiment 43, further comprising a first separator in fluid contact with the first reservoir and the first end, a second separator in fluid contact with the second reservoir and the second end, or a combination thereof.

### Embodiment 45

The device of embodiment 44, wherein the first separator is located between the first reservoir and the first end, the second separator is located between the second reservoir and the second end, or a combination thereof.

### Embodiment 46

The device of any one of embodiments 42-45, further comprising a slip layer, wherein the slip layer comprises a fluid permeable region defined by a fluid impermeable boundary, wherein the slip layer can be translocated from a retracted position to a deployed position, wherein in the retracted position the fluid permeable region of the slip layer is fluidly independent from the fluid permeable column, and wherein in the deployed position, the fluid permeable region of the slip layer is in fluid contact with the fluid permeable column.

### Embodiment 47

The device of any one of embodiments 42-46, wherein the plurality of planar segments are joined together in a sheet, and the plurality of planar segments are stacked by folding the sheet.

### Embodiment 48

The device of embodiment 47, wherein folding the sheet comprises accordion folding the sheet.

### Embodiment 49

The device of any one of embodiments 42-48, wherein the plurality of planar segments comprises at least 3 planar segments.

### Embodiment 50

The device of any one of embodiments 42-49, wherein the fluid permeable column is 10 mm or less in length.

### Embodiment 51

The device of any one of embodiments 42-50, wherein the device is paper based.

### Embodiment 52

The device of any one of embodiments 42-51, wherein the device further comprises a housing.

### Embodiment 53

A method comprising:

introducing a sample to the fluid permeable column of the device of any one of embodiments 43-52; and

applying a potential to the fluid permeable column.

### Embodiment 54

The method of embodiment 53, wherein the potential is 40 V or less.

### Embodiment 55

The method of any one of embodiments 53-54, wherein the sample comprises an analyte.

### Embodiment 56

The method of embodiment 55, further comprising separating the analyte from the sample.

### Embodiment 57

The method of any one of embodiments 53-56, further comprising accumulating the sample, the analyte, or a combination thereof in a section of the fluid permeable column.

### Embodiment 58

The method of embodiment 57, further comprising removing the section of the fluid permeable column to isolate the sample, the analyte, or a combination thereof.

### Embodiment 59

The method of any one of embodiments 57-58, wherein the section can comprise one or more of the planar segments, a slip layer, or a combination thereof.

### Embodiment 60

The method of any one of embodiments 53-59, further comprising analyzing the sample, the analyte, or a combination thereof to determine a property of the sample, the analyte, or a combination thereof.

### Embodiment 61

The method of any one of embodiments 53-60, wherein introducing the sample to the fluid permeable column comprises translocating the slip layer to the deployed position, wherein the sample is initially located in the fluid permeable region of the slip layer.

### Embodiment 62

A device comprising:

a stack formed from a plurality of parallel segments;

a first fluid permeable column traversing the stack from a first end to a second end;

a second fluid permeable column traversing the stack from a third end to a fourth end;

a first electrode in electrical contact with the first end, the third end, or a combination thereof; and

a second electrode in electrical contact with the second end, the fourth end, or a combination thereof;

wherein each segment comprises a first fluid permeable region defined by a first fluid impermeable boundary and a second fluid permeable region defined by a second fluid impermeable boundary; and

wherein stacking of the plurality of segments aligns the first fluid permeable region within each of the plurality of parallel segments to form the first fluid permeable column and the second fluid permeable region within each of the plurality of parallel segments to form the second fluid permeable column.

### Embodiment 63

The device of embodiment 62, further comprising:

a first reservoir in fluid contact with the first end, the third end, or a combination thereof;

a second reservoir in fluid contact with the second end, the fourth end, or a combination thereof; or

a combination thereof

### Embodiment 64

The device of embodiment 63, further comprising:

a first separator in fluid contact with the first reservoir and the first end, the third end, or a combination thereof;

a second separator in fluid contact with the second reservoir and the second end, the fourth end, or a combination thereof; or

a combination thereof.

### Embodiment 65

The device of embodiment 64, wherein

the first separator is located between the first reservoir and the first end, the third end, or a combination thereof;

the second separator is located between the second reservoir and the second end, the fourth end, or a combination thereof; or

a combination thereof.

### Embodiment 66

The device of any one of embodiments 62-65, further comprising a loading slip layer, wherein the loading slip layer comprises:

a first fluid permeable region defined by a first fluid impermeable boundary;

wherein the loading slip layer can be translocated from a refracted position to a deployed position;

wherein in the retracted position the first fluid permeable region of the loading slip layer is fluidly independent from the first fluid permeable column and the second fluid permeable column; and

wherein in the deployed position, the first fluid permeable region of the loading slip layer is in fluid contact with the first fluid permeable column, the second fluid permeable column, or a combination thereof.

### Embodiment 67

The device of embodiment 66, wherein the loading slip layer further comprises a second fluid permeable region defined by a second fluid impermeable boundary;

wherein in the retracted position the second fluid permeable region of the loading slip layer is fluidly independent from the first fluid permeable column and the second fluid permeable column; and

wherein in the deployed position, the second fluid permeable region of the loading slip layer is in fluid contact with the second fluid permeable column.

### Embodiment 68

The device of any one of embodiments 66-67, further comprising a collection slip layer, wherein the collection slip layer comprises:

a fluid permeable region defined by a fluid impermeable boundary;

wherein the collection slip layer can be translocated from a first position to a second position;

wherein in the first position, the fluid permeable region of the collection slip layer is in fluid contact with the first fluid permeable column and fluidly independent from the second fluid permeable column; and

wherein in the second position, the fluid permeable region of the collection slip layer is in fluid contact with the second fluid permeable column and fluidly independent from the first fluid permeable column.

### Embodiment 69

The device of any one of embodiments 62-68, wherein the plurality of segments are joined together in a sheet, and the stack is formed by folding the sheet.

### Embodiment 70

The device of embodiment 69, wherein folding the sheet comprises accordion folding the sheet.

### Embodiment 71

The device of any one of embodiments 62-70, wherein the plurality of parallel segments comprises at least 3 parallel segments.

### Embodiment 72

The device of any one of embodiments 62-71, wherein the first fluid permeable column, the second fluid permeable column, or a combination thereof is 10 mm or less in length.

### Embodiment 73

The device of any one of embodiments 62-72, wherein the device is paper based.

### Embodiment 74

The device of any one of embodiments 62-73, wherein the device further comprises a housing.

### Embodiment 75

A method comprising:

introducing a sample to the first fluid permeable column, the second fluid permeable column, or a combination thereof of the device of any one of embodiments 62-74; and

applying a potential to the first fluid permeable column, the second fluid permeable column, or a combination thereof.

### Embodiment 76

The method of embodiment 75, wherein the potential is 40 V or less.

### Embodiment 77

The method of any one of embodiments 75-76, wherein the sample comprises a first analyte.

### Embodiment 78

The method of embodiment 77, further comprising separating the first analyte from the sample.

### Embodiment 79

The method of any one of embodiments 75-78, further comprising accumulating the sample, the first analyte, or a combination thereof in a section of the first fluid permeable column, the second fluid permeable column, or a combination thereof.

### Embodiment 80

The method of embodiment 79, further comprising removing the section of the first fluid permeable column, the second fluid permeable column, or a combination thereof to isolate the sample, the first analyte, or a combination thereof.

### Embodiment 81

The method of any one of embodiments 79-80, wherein the section comprises one or more of the parallel segments, a loading slip layer, a collection slip layer, or a combination thereof

### Embodiment 82

The method of any one of embodiments 75-81, further comprising analyzing the sample, the first analyte, or a combination thereof to determine a property of the sample, the first analyte, or a combination thereof.

### Embodiment 83

The method of any one of embodiments 75-82, wherein introducing the sample to the first fluid permeable column, the second fluid permeable column, or a combination thereof comprises translocating the loading slip layer to the deployed position, wherein the sample is initially located in the first fluid permeable region of the loading slip layer, the second fluid permeable region of the loading slip layer, or a combination thereof.

### Embodiment 84

The method of embodiment 79, wherein the section of the first fluid permeable column comprises the collection slip layer in the first position.

### Embodiment 85

The method of embodiment 84, further comprising translocating the collection slip layer to the second position.

### Embodiment 86

The method of embodiment 85, further comprising applying a potential to the second fluid permeable column.

### Embodiment 87

The method of embodiment 86, wherein the potential is 40 V or less.

### Embodiment 88

The method of any one of embodiments 84-87, wherein the sample, the first analyte, or a combination thereof further comprises a second analyte.

### Embodiment 89

The method of embodiment 88, further comprising separating the second analyte from the sample, the first analyte, or a combination thereof.

### Embodiment 90

The method of any one of embodiments 85-89, further comprising accumulating the sample, the first analyte, the second analyte or a combination thereof in a section of the second fluid permeable column.

### Embodiment 91

The method of embodiment 90, further comprising removing the section of the second fluid permeable column to isolate the sample, the first analyte, the second analyte, or a combination thereof.

### Embodiment 92

The method of any of embodiments 90-91, wherein the section of the second fluid permeable column comprises one or more of the parallel segments, the loading slip layer, the collection slip layer, or a combination thereof.

### Embodiment 93

The method of any one of embodiments 85-92, further comprising analyzing the sample, the first analyte, the second analyte, or a combination thereof to determine a property of the sample, the first analyte, the second analyte, or a combination thereof.

### Embodiment 94

The method of any one of embodiments 85-93, wherein introducing the sample to the first fluid permeable column comprises translocating the loading slip layer to the deployed position, wherein the sample is initially located in the first fluid permeable region of the loading slip layer, the second fluid permeable region of the loading slip layer, or a combination thereof.

### Embodiment 95

A device comprising:

a plurality of planar segments, each planar segment comprising a first fluid permeable region defined by a first fluid impermeable boundary and a second fluid permeable region defined by a second fluid impermeable boundary;

wherein when the plurality of planar segments are stacked such that the first fluid permeable region of each planar segment is aligned and the second fluid permeable region of each planar segment is aligned, the first fluid permeable regions of the plurality of planar segments collectively forms a first fluid permeable column traversing the stacked plurality of planar segments from a first end to a second end, and the second fluid permeable regions of the plurality of planar segments collectively forms a second fluid permeable column traversing the stacked plurality of planar segments from a third end to a fourth end.

### Embodiment 96

The device of embodiment 95, further comprising:

a first electrode in electrical contact with the first end, the third end, or a combination thereof;

a second electrode in electrical contact with the second end, the fourth end, or a combination thereof; or

a combination thereof.

### Embodiment 97

The device of any one of embodiments 95-96, further comprising:

a first reservoir in fluid contact with the first end, the third end, or a combination thereof;

a second reservoir in fluid contact with the second end, the fourth end, or a combination thereof; or

a combination thereof.

### Embodiment 98

The device of embodiment 97, further comprising:

a first separator in fluid contact with the first reservoir and the first end, the third end, or a combination thereof;

a second separator in fluid contact with the second reservoir and the second end, the fourth end, or a combination thereof; or

a combination thereof.

### Embodiment 99

The device of embodiment 98, wherein

the first separator is located between the first reservoir and the first end, the third end, or a combination thereof;

the second separator is located between the second reservoir and the second end, the fourth end, or a combination thereof; or

a combination thereof.

### Embodiment 100

The device of any one of embodiments 95-99, further comprising a loading slip layer, wherein the loading slip layer comprises:

a first fluid permeable region defined by a first fluid impermeable boundary;

wherein the loading slip layer can be translocated from a refracted position to a deployed position;

wherein in the retracted position the first fluid permeable region of the loading slip layer is fluidly independent from the first fluid permeable column and the second fluid permeable column; and

wherein in the deployed position, the first fluid permeable region of the loading slip layer is in fluid contact with the first fluid permeable column, the second fluid permeable column, or a combination thereof.

### Embodiment 101

The device of embodiment 100, wherein the loading slip layer further comprises:

a second fluid permeable region defined by a second fluid impermeable boundary;

wherein in the retracted position the second fluid permeable region of the loading slip layer is fluidly independent from the first fluid permeable column and the second fluid permeable column; and

wherein in the deployed position, the second fluid permeable region of the loading slip layer is in fluid contact with the second fluid permeable column.

### Embodiment 102

The device of any one of embodiments 100-101, further comprising a collection slip layer, wherein the collection slip layer comprises:

a fluid permeable region defined by a fluid impermeable boundary;

wherein the collection slip layer can be translocated from a first position to a second position;

wherein in the first position, the fluid permeable region of the collection slip layer is in fluid contact with the first fluid permeable column and fluidly independent from the second fluid permeable column; and

wherein in the second position, the fluid permeable region of the collection slip layer is in fluid contact with the second fluid permeable column and fluidly independent from the first fluid permeable column.

### Embodiment 103

The device of any one of embodiments 95-102, wherein the plurality of planar segments are joined together in a sheet, and the plurality of planar segments is stacked by folding the sheet.

### Embodiment 104

The device of embodiment 103, wherein folding the sheet comprises accordion folding the sheet.

### Embodiment 105

The device of any one of embodiments 95-104, wherein the plurality of planar segments comprises at least 3 planar segments.

### Embodiment 106

The device of any one of embodiments 95-105, wherein the first fluid permeable column, the second fluid permeable column, or a combination thereof is 10 mm or less in length.

### Embodiment 107

The device of any one of embodiments 95-106, wherein the device is paper based.

### Embodiment 108

The device of any one of embodiments 95-107, wherein the device further comprises a housing.

### Embodiment 109

A method comprising:

introducing a sample to the first fluid permeable column, the second fluid permeable column, or a combination thereof of the device of any one of embodiments 95-108; and

applying a potential to the first fluid permeable column, the second fluid permeable column, or a combination thereof.

### Embodiment 110

The method of embodiment 109, wherein the potential is 40 V or less.

### Embodiment 111

The method of any one of embodiments 109-110, wherein the sample comprises a first analyte.

### Embodiment 112

The method of embodiment 111, further comprising separating the first analyte from the sample.

### Embodiment 113

The method of any one of embodiments 109-112, further comprising accumulating the sample, the first analyte, or a combination thereof in a section of the first fluid permeable column, the second fluid permeable column, or a combination thereof.

### Embodiment 114

The method of embodiment 113, further comprising removing the section of the first fluid permeable column, the second fluid permeable column, or a combination thereof to isolate the sample, the first analyte, or a combination thereof.

### Embodiment 115

The method of any one of embodiments 113-114, wherein the section comprises one or more of the parallel segments, the loading slip layer, the collection slip layer, or a combination thereof.

### Embodiment 116

The method of any one of embodiments 109-115, further comprising analyzing the sample, the first analyte, or a combination thereof to determine a property of the sample, the first analyte, or a combination thereof.

### Embodiment 117

The method of any one of embodiments 109-116, wherein introducing the sample to the first fluid permeable column, the second fluid permeable column, or a combination thereof comprises translocating the loading slip layer to the deployed position, wherein the sample is initially located in the first fluid permeable region of the loading slip layer, the second fluid permeable region of the loading slip layer, or a combination thereof.

### Embodiment 118

The method of embodiment 113, wherein the section of the first fluid permeable column comprises the collection slip layer in the first position.

### Embodiment 119

The method of embodiment 118, further comprising translocating the collection slip layer to the second position.

### Embodiment 120

The method of embodiment 119, further comprising applying a potential to the second fluid permeable column.

### Embodiment 121

The method of embodiment 120, wherein the potential is 40 V or less.

### Embodiment 122

The method of any one of embodiments 118-121, wherein the sample, the first analyte, or a combination thereof further comprises a second analyte.

### Embodiment 123

The method of embodiment 122, further comprising separating the second analyte from the sample, the first analyte, or a combination thereof.

### Embodiment 124

The method of any one of embodiments 119-123, further comprising accumulating the sample, the first analyte, the second analyte or a combination thereof in a section of the second fluid permeable column.

### Embodiment 125

The method of embodiment 124, further comprising removing the section of the second fluid permeable column to isolate the sample, the first analyte, the second analyte, or a combination thereof.

### Embodiment 126

The method of any of embodiments 124-125, wherein the section of the second fluid permeable column comprises one or more of the parallel segments, the loading slip layer, the collection slip layer, or a combination thereof.

### Embodiment 127

The method of any one of embodiments 118-126, further comprising analyzing the sample, the first analyte, the second analyte, or a combination thereof to determine a property of the sample, the first analyte, the second analyte, or a combination thereof.

### Embodiment 128

The method of any one of embodiments 118-127, wherein introducing the sample to the first fluid permeable column comprises translocating the loading slip layer to the deployed position, wherein the sample is initially located in the first fluid permeable region of the loading slip layer, the second fluid permeable region of the loading slip layer, or a combination thereof.

### Embodiment 129

A device comprising:

a plurality of planar segments, each planar segment comprising:

a top surface;

a bottom surface; and

a first fluid permeable region defined by a first fluid impermeable boundary extending through the planar segment from the top surface to the bottom surface so as to form a first fluid permeable pathway extending through the planar segment from the top surface to the bottom surface;

a second fluid permeable region defined by a second fluid impermeable boundary extending through the planar segment from the top surface to the bottom surface so as to form a second fluid permeable pathway extending through the planar segment from the top surface to the bottom surface;

wherein when the plurality of segments are stacked such that the bottom surface of a first planar segment is in intimate contact with the top surface of a second planar segment:

the first fluid permeable regions together form a first fluid permeable column within the stacked plurality of planar segments extending from a first end to a second end; and

the second fluid permeable regions together form a second fluid permeable column within the stacked plurality of planar segments extending from a third end to a fourth end;

wherein the first end comprises the first fluid permeable region at the top surface of the first planar segment;

wherein the second end comprises the first fluid permeable region at the bottom surface of the last planar segment;

wherein the third end comprises the second fluid permeable region at the top surface of the first planar segment;

wherein the fourth end comprises the second fluid permeable region at the bottom surface of the last planar segment;

a first electrode in electrical contact with the first end, the third end, or a combination thereof;

a second electrode in electrical contact with the second end, the fourth end, or a combination thereof.

### Embodiment 130

The device of embodiment 129, further comprising:

a first reservoir in fluid contact with the first end, the third end, or a combination thereof;

a second reservoir in fluid contact with the second end, the fourth end, or a combination thereof; or

a combination thereof.

### Embodiment 131

The device of embodiment 130, further comprising:

a first separator in fluid contact with the first reservoir and the first end, the third end, or a combination thereof;

a second separator in fluid contact with the second reservoir and the second end, the fourth end, or a combination thereof; or

a combination thereof.

### Embodiment 132

The device of embodiment 131, wherein

the first separator is located between the first reservoir and the first end, the third end, or a combination thereof;

the second separator is located between the second reservoir and the second end, the fourth end, or a combination thereof; or

a combination thereof.

### Embodiment 133

The device of any one of embodiments 129-132, further comprising a loading slip layer, wherein the loading slip layer comprises:

a first fluid permeable region defined by a first fluid impermeable boundary;

wherein the loading slip layer can be translocated from a refracted position to a deployed position;

wherein in the retracted position the first fluid permeable region of the loading slip layer is fluidly independent from the first fluid permeable column and the second fluid permeable column; and

wherein in the deployed position, the first fluid permeable region of the loading slip layer is in fluid contact with the first fluid permeable column, the second fluid permeable column, or a combination thereof

### Embodiment 134

The device of embodiment 133, wherein the loading slip layer further comprises:

a second fluid permeable region defined by a second fluid impermeable boundary;

wherein in the retracted position the second fluid permeable region of the loading slip layer is fluidly independent from the first fluid permeable column and the second fluid permeable column; and

wherein in the deployed position, the second fluid permeable region of the loading slip layer is in fluid contact with the second fluid permeable column.

### Embodiment 135

The device of any one of embodiments 133-134, further comprising a collection slip layer, wherein the collection slip layer comprises:

a fluid permeable region defined by a fluid impermeable boundary;

wherein the collection slip layer can be translocated from a first position to a second position;

wherein in the first position, the fluid permeable region of the collection slip layer is in fluid contact with the first fluid permeable column and fluidly independent from the second fluid permeable column; and

wherein in the second position, the fluid permeable region of the collection slip layer is in fluid contact with the second fluid permeable column and fluidly independent from the first fluid permeable column.

### Embodiment 136

The device of any one of embodiments 129-135, wherein the plurality of planar segments comprises a planar sheet and the plurality of planar segments is stacked by folding the planar sheet.

### Embodiment 137

The device of embodiment 136, wherein folding the planar sheet comprises accordion folding the planar sheet.

### Embodiment 138

The device of any one of embodiments 129-137, wherein the plurality of planar segments comprises 3 planar segments or more.

### Embodiment 139

The device of any one of embodiments 129-138, wherein the first fluid permeable column, the second fluid permeable column, or a combination thereof is 10 mm or less in length.

### Embodiment 140

The device of any one of embodiments 129-139, wherein the device is paper based.

### Embodiment 141

The device of any one of embodiments 129-140, wherein the device further comprises a housing.

### Embodiment 142

A method comprising:

introducing a sample to the first fluid permeable column, the second fluid permeable column, or a combination thereof of the device of any one of embodiments 129-141; and

applying a potential to the first fluid permeable column, the second fluid permeable column, or a combination thereof.

### Embodiment 143

The method of embodiment 142, wherein the potential is 40 V or less.

### Embodiment 144

The method of any one of embodiments 142-143, wherein the sample comprises a first analyte.

### Embodiment 145

The method of embodiment 144, further comprising separating the first analyte from the sample.

### Embodiment 146

The method of any one of embodiments 142-145, further comprising accumulating the sample, the first analyte, or a combination thereof in a section of the first fluid permeable column, the second fluid permeable column, or a combination thereof.

### Embodiment 147

The method of embodiment 146, further comprising removing the section of the first fluid permeable column, the second fluid permeable column, or a combination thereof to isolate the sample, the first analyte, or a combination thereof.

### Embodiment 148

The method of any one of embodiments 146-147, wherein the section comprises one or more of the parallel segments, the loading slip layer, the collection slip layer, or a combination thereof.

### Embodiment 149

The method of any one of embodiments 142-148, further comprising analyzing the sample, the first analyte, or a combination thereof to determine a property of the sample, the first analyte, or a combination thereof.

### Embodiment 150

The method of any one of embodiments 142-149, wherein introducing the sample to the first fluid permeable column, the second fluid permeable column, or a combination thereof comprises translocating the loading slip layer to the deployed position, wherein the sample is initially located in the first fluid permeable region of the loading slip layer, the second fluid permeable region of the loading slip layer, or a combination thereof.

### Embodiment 151

The method of embodiment 146, wherein the section of the first fluid permeable column comprises the collection slip layer in the first position.

### Embodiment 152

The method of embodiment 151, further comprising translocating the collection slip layer to the second position.

### Embodiment 153

The method of embodiment 152, further comprising applying a potential to the second fluid permeable column.

### Embodiment 154

The method of embodiment 153, wherein the potential is 40 V or less.

### Embodiment 155

The method of any one of embodiments 151-154, wherein the sample, the first analyte, or a combination thereof further comprises a second analyte.

### Embodiment 156

The method of embodiment 155, further comprising separating the second analyte from the sample, the first analyte, or a combination thereof

### Embodiment 157

The method of any one of embodiments 152-156, further comprising accumulating the sample, the first analyte, the second analyte or a combination thereof in a section of the second fluid permeable column.

### Embodiment 158

The method of embodiment 157, further comprising removing the section of the second fluid permeable column to isolate the sample, the first analyte, the second analyte, or a combination thereof.

### Embodiment 159

The method of any of embodiments 157-158, wherein the section of the second fluid permeable column comprises one or more of the parallel segments, the loading slip layer, the collection slip layer, or a combination thereof.

### Embodiment 160

The method of any one of embodiments 151-159, further comprising analyzing the sample, the first analyte, the second analyte, or a combination thereof to determine a property of the sample, the first analyte, the second analyte, or a combination thereof.

### Embodiment 161

The method of any one of embodiments 151-160, wherein introducing the sample to the first fluid permeable column comprises translocating the loading slip layer to the deployed position, wherein the sample is initially located in the first fluid permeable region of the loading slip layer, the second fluid permeable region of the loading slip layer, or a combination thereof

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process.

### Example 1

### Overview

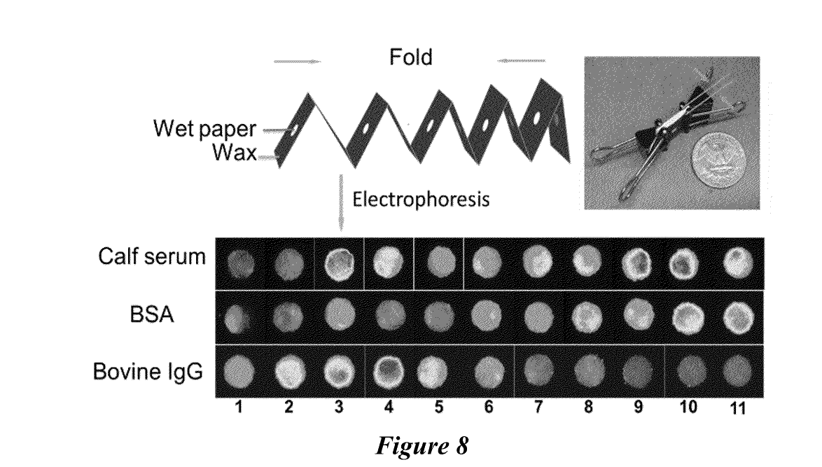

Presented herein is an origami paper-based electrophoretic device (oPAD-Ep), which can achieve rapid (˜5 min) separation of fluorescent molecules and proteins. The driving voltage was ˜10 V, which is more than 10 times lower than that used for conventional electrophoresis. The oPAD-Ep used multiple, thin (180 μm/layer) folded paper layers as the supporting medium for electrophoresis. This approach can shorten the distance between the anode and cathode, and this in turn can account for the high electric field (>1 kV/m) that can be achieved even with a low applied voltage. The multilayer design of the oPAD-Ep can enable sample introduction by use of a slip layer, as well as product analysis and reclamation after electrophoresis by unfolding the origami paper and cutting out desired layers. The use of oPAD-Ep for simple separation of proteins in bovine serum is demonstrated (FIG. 8), which indicates its potential applications for point-of-care diagnostic testing.

Introduction

Herein, an electrophoretic (Ep) device, which can be integrated into paper analytical devices (PADs) (Maxwell E J et al. MRS Bull. 2013, 38, 309-314; Martinez A W et al. Anal. Chem. 2010, 82, 3-10) is discussed, and separation of fluorescent molecules and proteins at low voltages is demonstrated. The device, which is referred to herein as an oPAD-Ep (the “o” stands for origami) (Liu H and Crooks R M. J. Am. Chem. Soc. 2011, 133, 17564-17566), can be easy to construct (FIG. 4). Briefly, a piece of filter paper was folded into a multilayer structure that can serve as the Ep medium. A slip layer can be added to introduce the sample (Liu H et al. Anal. Chem. 2013, 85, 4263-4267), and this assembly can then be sandwiched between two Ag/AgCl electrode assemblies. An electric field of a few kV/m can be generated in an oPAD-Ep using an applied voltage of 10 V due to the thinness of the folded paper (˜2 mm thick for an 11-layer origami construct). In contrast, a much higher applied voltage (100-300 V) can be required to achieve a similar field using a conventional Ep apparatus (Magdeldin S., Ed. Gel Electrophoresis—Principles and Basics. InTech: Croatia, 2012). In addition, a separate slip layer can be incorporated in oPAD-Ep for sample introduction (Liu H et al. Anal. Chem. 2013, 85, 4263-4267). The position of the slip layer can determine the initial location of sample. Product analysis after Ep can be performed by unfolding the device, and the resolution of product distribution can be as high as the thickness of a single paper layer (˜180 μm). After Ep separation, the paper can be cut to reclaim one or multiple components from a complex mixture for further analysis. The oPAD-Ep can provide an alternative means for transport of charged molecules through wetted paper when normal capillary driven flow is absent or too slow. The simple construction, low voltage requirement, and other properties alluded to above can make oPAD-Ep suitable for point-of-care (POC) applications; for example, as a component of diagnostic devices.

In the 1930s, Tiselius developed the first Ep system, the “Tiselius apparatus”, for analysis of colloidal mixtures (Tiselius A. Trans. Faraday Soc. 1937, 33, 0524-0530). This technique has evolved over time to take advantage of physical and chemical differences between targets (such as proteins or DNA). For example, the supporting medium may be filter paper, natural gels, or synthetic gels (Martin N H and Franglen G T. J. Clin. Pathol. 1954, 7, 87-105; Scopes R K. Biochem. J. 1968, 107, 139-150; Thorne H V. Virology 1966, 29, 234-239; Meyers J A et al. J. Bacteriol. 1976, 127, 1529-1537; Giri K V. Nature 1957, 179, 632-632; Bachvaroff R and McMaster P R. Science 1964, 143, 1177-1179; Chrambach A and Rodbard D. Science 1971, 172, 440-451). The apparatuses used to carry out these separations can also vary widely, for example SDS-PAGE, capillary Ep, and isoelectric focusing (Weber K and Osborn M. J. Biol. Chem. 1969, 244, 4406-4412; Schägger H and Von Jagow G. Anal. Biochem. 1987, 166, 368-379; Pedersen-Bjergaard S and Rasmussen K E. Anal. Chem. 1999, 71, 2650-2656; Neuhoff V et al. Electrophoresis 1988, 9, 255-262, Bjellqvist B et al. J. Biochem. Bioph. Methods 1982, 6, 317-339).

In recent years, simple forms of paper Ep have been developed that can be incorporated into POC devices. For example, Ge et al. introduced a paper-based electrophoretic device for amino acid separation by imitating the design of conventional electrophoretic systems (Ge L et al. Chem. Commun. 2014, 50, 5699-5702). Using wax printing (Carrilho E et al. Anal. Chem. 2009, 81, 7091-7095), they patterned two reservoirs connected by a ˜20 mm-long channel on paper. A voltage of 330 V was applied across the channel, which achieved an electro-migration speed of a few mm/min for amino acids. Using an alternative design, Chen et al. achieved a similar electric field, but avoided the necessity of using a high applied voltage by placing the anode and cathode in close proximity (˜2 mm) (Chen S S et al. Lab Chip 2014, 14, 2124-2130). However, the device designs mentioned above involve either a high voltage, which is not suitable for POC applications, or challenging operational characteristics. Moreover, a constant pH was not maintained in either of these two devices, raising concerns about nonuniform Ep of amphoteric molecules, whose mobilities can be strongly dependent on the solution pH. The multilayer oPAD-Ep design described herein addresses these types of issues.

Three-dimensional (3D) PADs were first reported by Whitesides and coworkers in 2008 (Martinez A W et al. Proc. Natl. Acad. Sci. 2008, 105, 19606-19611). In these devices, multiple paper layers were stacked and held together with double-sided tape. More recently, a simpler method for achieving similar functionality was introduced by using the fabrication principles of origami; that is, folding a single piece of paper into a 3D geometry (Liu H and Crooks R M. J. Am. Chem. Soc. 2011, 133, 17564-17566). This family of sensors is referred to herein as oPADs. Since their inception, a number of oPADs have been reported for various applications, including: detection of biomolecules, paper-based batteries, and a microscope (Liu H et al. Angew. Chem., Int. Ed. 2012, 51, 6925-6928; Scida K et al. Anal. Chem. 2013, 85, 9713-9720; Ge L et al. Lab Chip 2012, 12, 3150-3158; Chen S S et al. Lab Chip 2014, 14, 2124-2130; Cybulski J S et al. PLoS ONE 2014, 9, e98781). In contrast to earlier systems, the oPAD-Ep takes advantage of the thinness of the paper used for device fabrication. This can result in a short distance between the anode and cathode (˜a few millimeters), which can lead to electric fields of ˜2 kV/m with an input voltage of 10 V. When subjected to this field, fluorescent molecules or proteins can penetrate each paper layer at a speed of 1-3 layers/min. Herein, the fundamental characteristics of the oPAD-Ep design are discussed, the separation of fluorescent molecules based on their different electrophoretic mobilities is demonstrated, and it is shown that bovine serum albumin (BSA) can be separated from calf serum within 5 min.

### Experimental

### Chemicals and Materials

Tris-HCl buffer (1.0 M, pH=8.0), phosphate buffered saline (PBS, pH=7.4), and Whatman Grade 1 chromatography paper, were purchased from Fisher Scientific. Silver wire (2.0 mm in diameter), calf serum from formula-fed bovine calves, albumin (lyophilized powder, ≧95%, agarose gel Ep) and IgG (reagent grade, ≧95%, SDS-PAGE, essentially salt-free, lyophilized powder) from bovine serum, and FluoroProfile protein quantification kits were purchased from Sigma-Aldrich. The following fluorescent molecules were used as received: Ru(bpy)₃Cl₂ (Fluka), 4,4-difluoro-1,3,5,7,8-pentamethyl-4-bora-3a,4a-diaza-s-indacene (BODIPY²⁻, Invitrogen), 8-methoxypyrene-1,3,6-trisulfonic acid trisodium salt (MPTS³⁻, Anaspec), 1,3,6,8-pyrenetetrasulfonic acid tetrasodium salt (PTS⁴⁻, Fisher Scientific), Rhodamine 6G (Acros), methylene blue (Sigma-Aldrich), and rhodamine B (Fluka). All solutions were prepared using deionized water having a resistivity of 18.2 MSΩ·cm from a Milli-Q Gradient System (Bedford, Mass.). Serum protein solutions were prepared with PBS.

Device Fabrication.

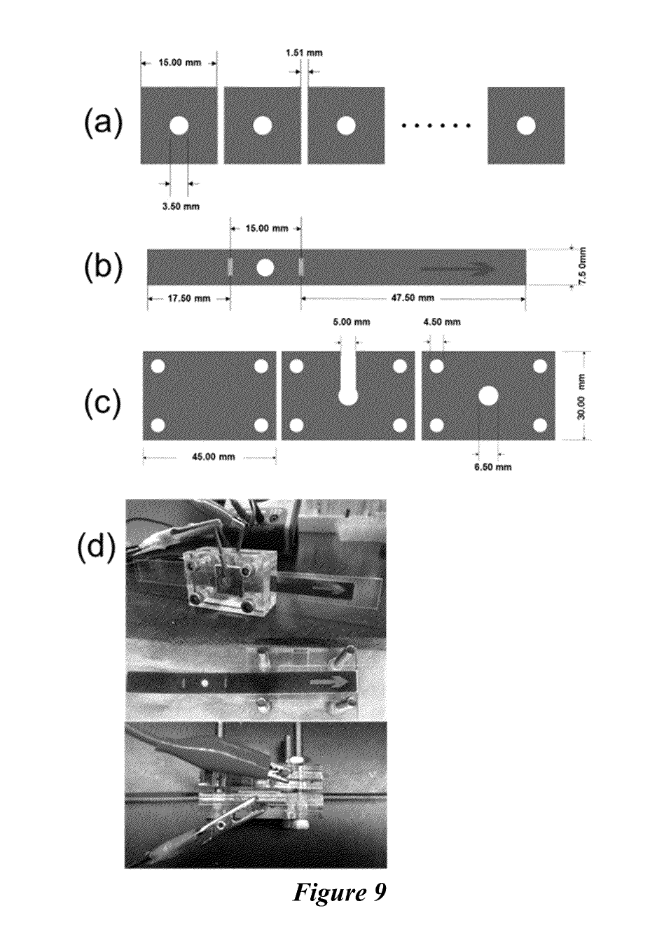

oPAD-Eps were fabricated in three steps: (1) the slip layer and origami paper were patterned using wax printing (Carrilho E et al. Anal. Chem. 2009, 81, 7091-7095), (2) the plastic buffer reservoirs were fabricated using a laser cutter, and (3) the oPAD-Eps were assembled as shown in FIG. 4. Briefly, CorelDraw software was used to design wax patterns on the Whatman Grade 1 paper (the patterns used for the origami sections and slip layers are shown in FIGS. 9a and b). After wax patterning using a Xerox 8570DN inkjet printer, the paper was placed in an oven at 120° C. for 45 seconds, and then cooled to 25±2° C. The unwaxed disk in the center of each section of the origami paper was ˜3.5 mm in diameter.

As shown in FIG. 9d, slip layers were partially laminated using Scotch self-sealing laminating pouches from 3M. The plastic sheath can reduce the friction between the slip layer and the wetted origami paper so that slipping does not cause damage to the paper. The plastic sheath can also ensure alignment of the sample loading zone on the slip layer with the channel in the origami paper. Similarly, fabrication of buffer reservoirs begins with a design in CorelDraw (FIG. 9c). Each reservoir consists of three layers, which are aligned, stacked, and then bound by acrylic adhesive (Weld-On). Each layer was fabricated by cutting a clear 0.32 cm-thick acrylic sheet using an Epilog Zing 16 laser cutter (Epilog Laser, Golden, Colo.). The layer in direct contact with the origami paper has a 6.5 mm-diameter hole at its center, and this was filled with a 5.0% agar gel prepared with buffer solution. This gel serves as a separator between the origami paper and reservoir solution, and it prevents the paper from being damaged by long-term exposure to solution, undesirable pH changes, and the effects of pressure-driven flow. After all parts were fabricated, they were assembled into the final device (FIG. 4 and FIG. 9d). Finally, the origami paper was pre-wetted with buffer solution, and the slip layer was placed in the desired position. The pressure holding the oPAD-Ep together is adjustable using four screws at the corners of the plastic sheets (FIG. 9d). Finger-tight torque was used for the experiments discussed herein.

Operation of the oPAD-Ep.

Before use, the two reservoirs of the oPAD-Ep were filled with 300.0 μL of buffer and then a Ag/AgCl electrode was inserted into each of them. For fluorescent molecule Ep, 0.20 M Tris-HCl (pH=8.0) was used, while PBS (pH=7.4) was used for protein Ep. Ag/AgCl electrodes were prepared by immersing Ag wires in commercial bleach overnight (Lathrop D K et al. J. Am. Chem. Soc. 2010, 132, 1878-1885), and rinsed thoroughly with DI water before use. The surface of the Ag wires turned dark brown after being oxidized to AgCl. A 0.50 μL aliquot of sample solution was loaded at the designated zone on the slip layer, and then introduced by pulling the slip layer into alignment with the origami paper. A BK Precision DC regulated power supply (model 1621A) was used to apply a voltage between the two Ag/AgCl electrodes. After Ep, the buffer and electrodes were removed from the reservoirs, and the screws were loosened to unfold the origami paper for analysis.

Fluorescence Analysis.

A Nikon AZ100 multi-purpose zoom fluorescence microscope was used to acquire fluorescent images of each oPAD-Ep layer, including the slip layer, and ImageJ software (NIH, Bethesda, Md.) was used to analyze the fluorescence intensity. For protein analyses, the FluoroProfile protein quantification kit from Sigma-Aldrich was used to label proteins with a fluorescent tag (Liu H and Crooks R M. J. Am. Chem. Soc. 2011, 133, 17564-17566). Following Ep, 0.50 μL FluoroProfile fluorescent reagent solution was spotted onto the origami paper and slip layer, both of which were placed in a humidity chamber for 30.0 min and then taken out and dried for an additional 30.0 min in a dark room. During this time period, epicocconone in the stain solution fully reacts with primary amine groups on proteins, producing a fluorescent conjugate having two excitation maxima at ˜400 nm and ˜500 nm, with emission at 610 nm (Bell P J and Karuso P. J. Am. Chem. Soc. 2003, 125, 9304-9305). An Omega XF204 filter (excitation: 540 nm and emission: 570-600 nm) was used to acquire the fluorescence images of stained proteins in the oPAD-Ep.

Results and Discussion

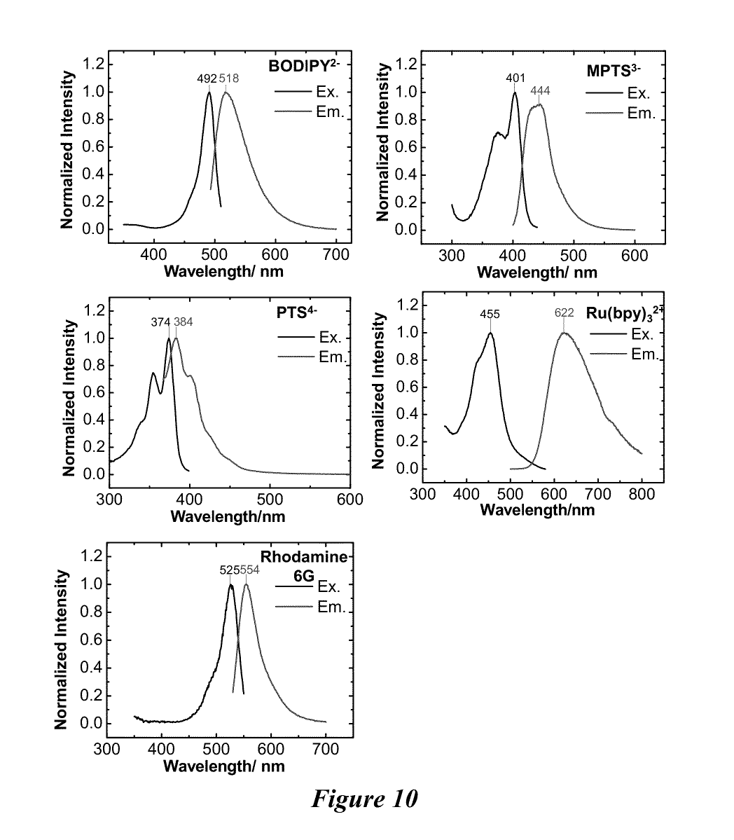

The Ep of single fluorescent molecules was examined, followed by the investigation of the use of the oPAD-Eps for more complex tasks including separation of fluorescent molecules and proteins. The fluorescent molecules used for demonstration purposes are listed in Table 1, along with their excitation and emission wavelengths (fluorescence spectra are provided in FIG. 10) and the corresponding microscope filter sets used for analysis.

TABLE 1
Spectral information about the fluorescence probes.

| | Absorption | Fluorescence | | |
|---|---|---|---|---|
| Fluorescent | Maximum | Maximum | Excitation | Emission |
| Molecules | Extinction (nm) | Emission (nm) | Filter (nm) | Filter (nm) |
| Ru(bpy)₃²⁺ | 455 | 622 | 420-490 | 510-700 |
| BODIPY²⁻ | 492 | 518 | 460-500 | 510-560 |
| MPTS³⁻ | 401 | 444 | 340-380 | 430-480 |
| PTS⁴⁻ | 374 | 384 | 340-380 | 430-480 |

For operation of the oPAD-Ep, the paper part of the device was folded, as shown in FIG. 4a, and then compressed by plastic sheets. The two reservoirs were filled with buffer and a Ag/AgCl electrode was inserted into each of them. Next, an aliquot of sample solution was loaded at the designated zone on the slip layer, and then the sample was introduced into the oPAD-Ep by pulling the slip layer into alignment with the origami paper. Finally, a voltage was applied to the electrodes until the separation was complete, at which time the origami paper was removed, unfolded, and analyzed.

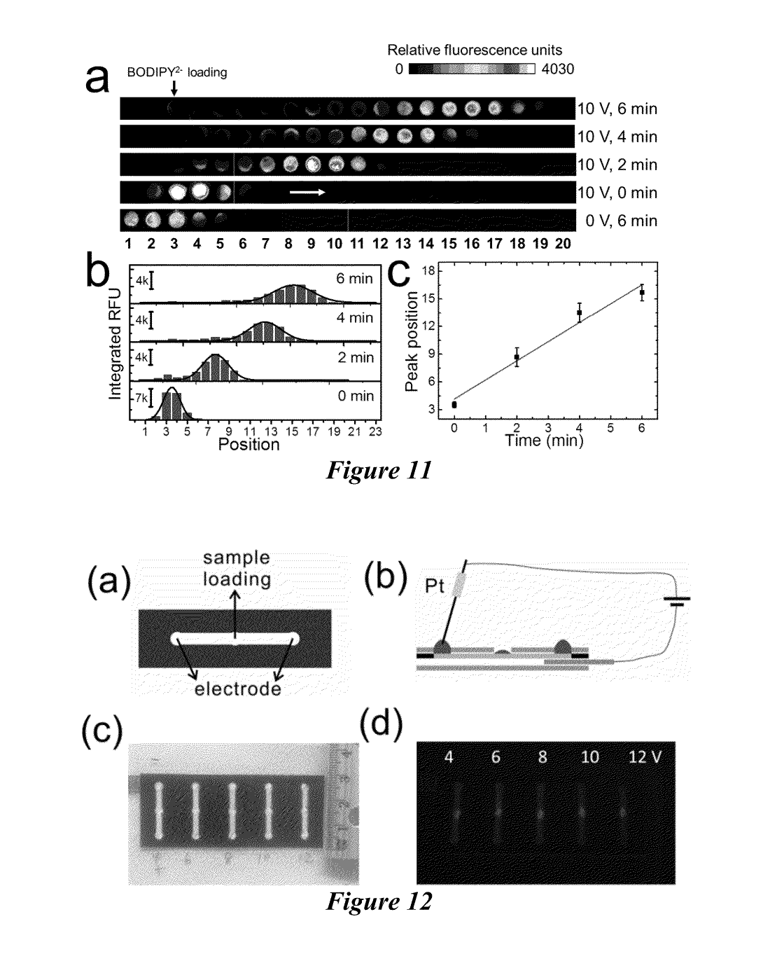

In a series of experiments, a 23-layer paper device was used to study the migration of BODIPY²⁻. This experiment was carried out by placing a 0.50 μL aliquot of 1.0 mM BODIPY²⁻ onto the slip layer, which was in turn placed between the second and third layers of the oPAD-Ep (e.g., Position 3 in FIG. 11). Upon application of 10.0 V, BODIPY²⁻ migrated from its initial location toward the cathode by penetrating each layer of the origami paper at a rate of ˜2-3 layers per min (FIG. 11). The distribution of BODIPY²⁻ broadened as a function of separation time: the width of the band increased from ˜2 layers at 0 min to ˜5 layers at 6.0 min. In the absence of the electric field (bottom of FIG. 11a), the initial BODIPY²⁻ spot broadened by ˜1 layer after 6.0 min. For comparative purposes, an Ep experiment in a 2.0 cm-long regular paper channel using the same applied voltage was also performed. In this control experiment, the paper electrophoretic device comprised a ˜2 cm-long straight paper channel with an electrode placed at each end (FIG. 12a). To rule out the interference of surface flow, the paper channel was embedded in a self-laminating pouch (FIG. 12b). The sample was loaded at the midpoint of the buffer-wetted paper channel, and the applied voltage between the two electrodes was varied from 4.0 V to 12.0 V to drive the electro-migration of BODIPY²⁻. After 5.0 min, no movement of the sample was observed under a UV lamp (FIG. 12d). In this case, no Ep transport was observed due to the weak electric field.

To provide a more quantitative analysis of the BODIPY²⁻ migration experiment (FIG. 11a), the fluorescence intensity (in terms of relative fluorescence units, RFU) of each layer of the oPAD-Ep was determined using ImageJ software and then plotted as a function of position (FIG. 11b). The standard deviations (σ) and peak positions (μ₀) of the distribution were obtained by fitting the results to a Gaussian distribution (black curves in FIG. 11b). Assuming that diffusion is the major cause of peak broadening, the diffusivity of BODIPY²⁻ in wet paper (Dₚₐₚₑᵣ) can be roughly estimated using the 1D Einstein diffusion equation (equation 1).

Δσ²=2Dₚₐₚₑᵣt (1)

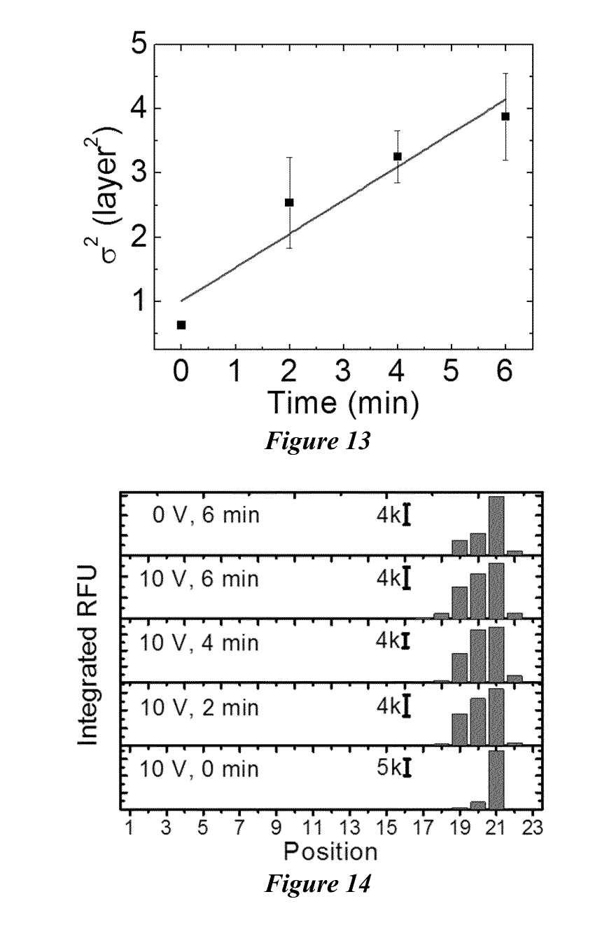

Here, Δσ² is the mean square displacement at time t. A plot of σ² vs. t is provided in FIG. 13, and from its slope Dₚₐₚₑᵣ was calculated to be ˜0.14×10⁻⁹ m²/s, which is about one third of the diffusivity of BODIPY²⁻ in water (D_{water}=˜0.43×10⁻⁹ m²/s) (Hlushkou D et al. Lab Chip 2009, 9, 1903-1913). This difference can be due to the presence of the network of cellulose fibers, which can hinder diffusion (Renkin E M. J. Gen. Physiol. 1954, 38, 225-243). The peak broadening exhibited by the fluorescent molecules in the oPAD-Ep can be caused by stochastic motion. Two additional points should be mentioned. First, the initial peak broadening observed at 0 min was caused by the sample transfer from the slip layer to the two neighboring layers. Second, there was little or no capillary flow in the oPAD-Ep, because all layers of the paper were pre-wetted with the running buffer prior to application of the applied voltage.

FIG. 11c shows that there is a linear correlation between the peak position (μ₀) and the time of Ep. The slope of this plot is 2.1 oPAD-Ep layers/min, which is equivalent to 6.0 μm/s. Using this value, equation 2 was used to estimate the Ep mobility (μ_{Ep}) of BODIPY²⁻ in wetted paper (E is the local electric field inside the device). To do so, a simplifying assumption, that Ep dominates electroosmosis under the conditions used in the experiments discussed herein, was made.

It has been shown previously that the electroosmotic velocity of albumin in barbital buffer in a variety of common papers ranges from ˜30% to 170% of its Ep velocity at pH 8.8 (Kunkel H G and Tiselius A. J. Gen. Physiol. 1951, 35, 89-118). In addition, Posner and coworkers observed significant electroosmotic flow (EOF) in nitrocellulose paper during their paper-based isotachophoretic preconcentration experiments. Specifically, they found that the fluorescent molecule AF488, which was focused between the leading and trailing electrolytes, moved faster (velocity increased from ˜30 μm/s to 150 μm/s) after adding 3% polyvinylpyrrolidone (PVP) to the leading electrolyte to suppress the EOF (Moghadam B Y et al. Anal. Chem. 2014, 86, 5829-5837). Clearly, the EOF in paper can vary over a wide range and can be dependent on experimental conditions such as paper structure and electrolyte. Therefore, the electroosmotic velocity of Rhodamine B, which is neutral in the pH range between 6.0 and 10.8 (Oh Y J et al. Lab Chip 2008, 8, 251-258), was measured to evaluate the EOF in oPAD-Eps. Rhodamine B (0.50 μL, 0.10 mM) was initially loaded onto the slip layer at Position 21, and the direction of applied electric field was from Position 23 toward Position 1 (FIG. 14); the running buffer was 0.20 M Tris-HCl (pH=8.0). The distributions of the integrated RFU for Rhodamine B in the 23-layer oPAD-Ep after Ep at an applied voltage of 10.0 V for run times ranging from 0 to 6.0 min and for 6.0 min with no applied voltage (top frame) are shown in FIG. 14. The results show that the electroosmotic velocity is small (<0.1 layer/min) compared to Ep, and therefore its contribution was ignored in the treatment shown in equation 2.

$${μ}_{Ep} = \frac{Δ {μ}_{0}}{E Δ t} \qquad ( 2 )$$

The following procedure was used to determine the value of E from equation 2. A multimeter was connected in series with the power supply to measure the current flowing through the oPAD-Ep with and without origami paper present in the device. At an applied voltage of 10.0 V, the values of the two currents were ˜1.7 mA and 6.0 mA, respectively. Using the difference between these currents and Ohm's law, the resistance of the origami paper was calculated to be ˜4.2 ka By multiplying this resistance by the current at 10.0 V, the voltage drop (ΔV) across the paper was determined to be ˜7 V. The value of E in the oPAD-Ep (˜1.7 kV/m at an applied voltage of 10.0 V) was then calculated by dividing ΔV by the total thickness (d=4.1 mm) of the 23-layer origami paper. Finally, using equation 2, μ_{Ep} for BODIPY²⁻ in the oPAD-Ep was calculated to be ˜2.2×10⁻⁹ m²/(s·V).

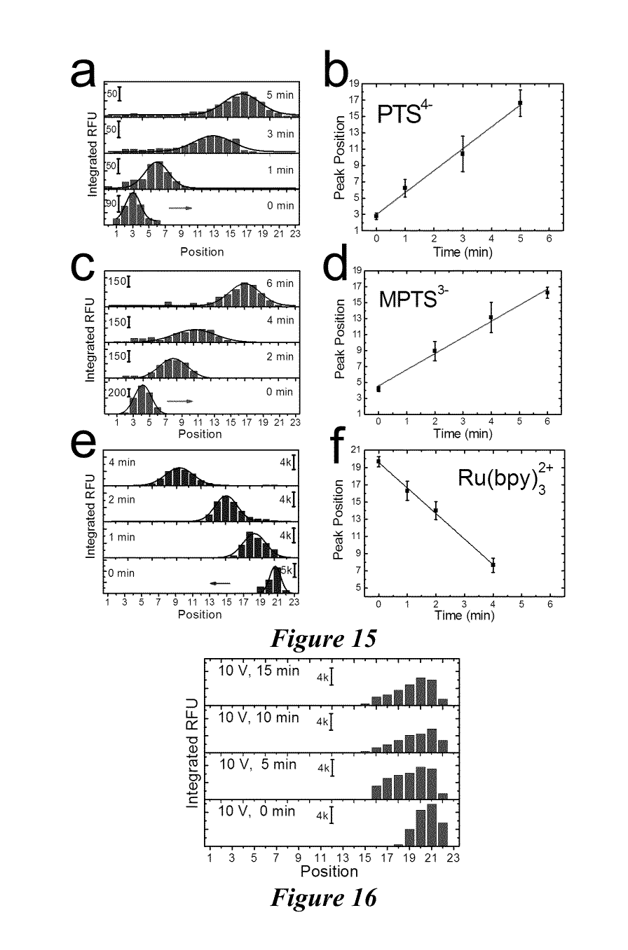

Following the procedure described for BODIPY²⁻, the Ep properties of three other dyes in the oPAD-Ep were evaluated: PTS⁴⁻, MPTS³⁻, and Ru(bpy)₃²⁺. Plots of the position of these dyes as a function of time are shown in FIG. 15. From these data, the Ep velocities were determined to be: PTS⁴⁻, 2.7 layers/min; MPTS³⁻, 2.0 layers/min; and Ru(bpy)₃²⁺, 3.0 layers/min. The corresponding values of μ_{Ep} are 2.9×10⁻⁹ m²/(s·V), 2.1×10⁻⁹ m²/(s·V), and 3.2×10⁻⁹ m²/(s·V), respectively. These mobilities are about one order of magnitude smaller than their counterparts in bulk solution (Laws D R et al. Anal. Chem. 2009, 81, 8923-8929; Wu J et al. Nat. Nanotechnol. 2012, 7, 133-139). There are several possible reasons for this: hindered migration by the cellulose matrix, specific interactions between the charged molecules and the paper, and small contributions arising from electroosmosis (the direction of EOF is opposite to the migrational direction of negatively charged dyes). Regardless of the underlying phenomena, the relative velocities are: Ru(bpy)₃²⁺>PTS⁴⁻>MPTS³⁻˜BODIPY²⁻. Two other positively charged dyes, Rhodamine 6G (+1 charge between pH 4.0 and 10.0; FIG. 16) (Milanova D et al. Electrophoresis 2011, 32, 3286-3294) and methylene blue were also tested, and both were found to migrate slowly (<0.2 layer/min) under the same conditions used for the other dyes. This may be a consequence of a strong electrostatic interaction between the negatively charged paper and the positively charged dyes.

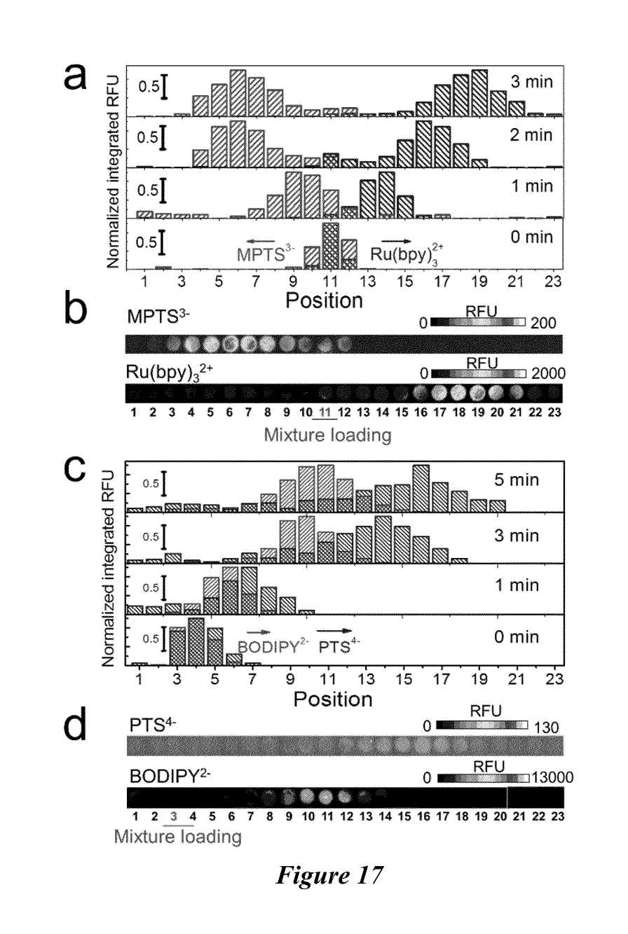

The separation of a mixture of two oppositely charged fluorescent molecules, MPTS³⁻ and Ru(bpy)₃²⁺, which migrate in opposite directions upon the application of an electric field, was examined using the oPAD-Ep. The separation of MPTS³⁻ and Ru(bpy)₃²⁺ was carried out as follows. A mixture containing 1.5 mM MPTS³⁻ and 1.5 mM Ru(bpy)₃²⁺ was prepared by mixing equal aliquots of 3.0 mM MPTS³⁻ and 3.0 mM Ru(bpy)₃²⁺. A 0.50 μL aliquot of the mixture was spotted onto the slip layer, and the slip layer was inserted at Position 11 of the oPAD-Ep. All other conditions were the same as in the previously described single-analyte experiments. When 10.0 V was applied between the two Ag/AgCl driving electrodes, MPTS³⁻ moved from its initial position towards the anode and Ru(bpy)₃²⁺ migrated toward the cathode. After carrying out the separation, each layer of the oPAD-Ep was characterized spectroscopically using a different fluorescence filter (Table 1). Because the emission intensity is different for the two dyes, the results of this experiment, shown in FIG. 17a, are normalized by setting the maximum RFU to 1. A near-quantitative separation was achieved in <1 min. FIG. 17b shows fluorescence images for the individual dyes (in the same oPAD-Ep) 3 min after the application of the voltage. Using the peak positions in FIG. 17b, the electrophoretic velocities are ˜2 and ˜3 layers/min for MPTS³⁻ and Ru(bpy)₃²⁺, respectively. These values are the same as those measured for the individual dyes.

The separation of two dyes with the same charge was also examined. This demonstration of the oPAD-Ep involved the separation of two negatively charged dyes, BODIPY²⁻ and PTS⁴⁻, which have the same charge but μ_{Ep} values that differ by about 25%. In this case, a 0.50 μL aliquot of a mixture containing 1.5 mM PTS⁴⁻ and 0.50 mM BODIPY²⁻ was initially situated at Position 3 (FIG. 17c). Upon application of 10.0 V, both molecules are driven toward the anode and gradually separate (FIG. 17c). The calculated Ep velocities of BODIPY²⁻ and PTS⁴⁻ (FIG. 11 and FIG. 15, respectively) are 2.1 and 2.7 layers/min. From these values, the predicted peak separation should be ˜3-4 paper layers after 5.0 min, which is in good agreement with the value of ˜5 layers found in the experiment (FIG. 17c). FIG. 17d shows fluorescence micrographs of BODIPY²⁻ and PTS⁴⁻ obtained in the same oPAD-Ep. The relatively low fluorescence intensity for PTS⁴⁻ in these experiments was caused by the small Stokes shift of this molecule which does not match perfectly with the fluorescence filter set used (Table 1 and FIG. 10).

Ep is widely used to separate biomolecules such as DNA and proteins. One of the most common electrophoretic techniques is gel Ep, which uses a gel to suppress the thermal convection caused by Joule heating and to sieve biomolecules on the basis of their size. This method is routinely used in clinical laboratories to test for abnormalities in a variety of biological matrices, including: serum, urine, blood, and cerebrospinal fluid (Jeppson J et al. Clin. Chem. 1979, 25, 629-638). For example, in serum protein gel Ep, normal serum is separated into five different bands: (1) Albumin, which is approximately two-thirds of the total protein content (3-5 g/dL); (2) Alpha-1 (0.1-0.3 g/dL) and (3) Alpha-2 (0.60-1.0 g/dL), which are two groups of globulins mainly including heptoglobin, ceruloplasmin, and macroglobin; (4) Beta (0.7-1.2 g/dL), composed of transferrin and lipoprotein; and (5) Gama (0.6-1.6 g/dL), which contains primarily immunogolublins such as IgG (Kyle R et al. Clinical Indications and Applications of Electrophoresis and Immunofixation. In Manual of Clinical Immunology; Rose N et al., Eds.; ASM Press: Washington D.C. 2002; pp 66-70). An excess or insufficiency in any of these bands can indicate a need for medical attention. Commercially available devices for separating serum proteins can require a high voltage (200-300 V) and a long separation time (˜1 h), both of which are impractical for POC applications. In this section, it is shown that the oPAD-Ep can rapidly (5 min) separate serum proteins using a voltage of 10V.

The Ep properties of bovine serum albumin (BSA) and IgG (also from bovine serum) were initially evaluated separately in the oPAD-Ep. In these experiments, an 11-layer oPAD-Ep was first wetted with 1×PBS buffer (ionic strength 163 mM, pH=7.4). Next, 0.50 μL of a 0.1×PBS buffer (ionic strength 16.3 mM) containing either 5.0 g/dL BSA or 1.0 g/dL IgG was loaded at Position 3 of the oPAD-Ep. These conditions are different from those used for separating the fluorescent molecules: the oPAD-Ep consists of fewer layers and the buffer concentration is lower, both of which serve to increase the electric field within the device. This experimental flexibility (e.g., the number of layers in the device) can be an advantageous feature of the oPAD-Ep.

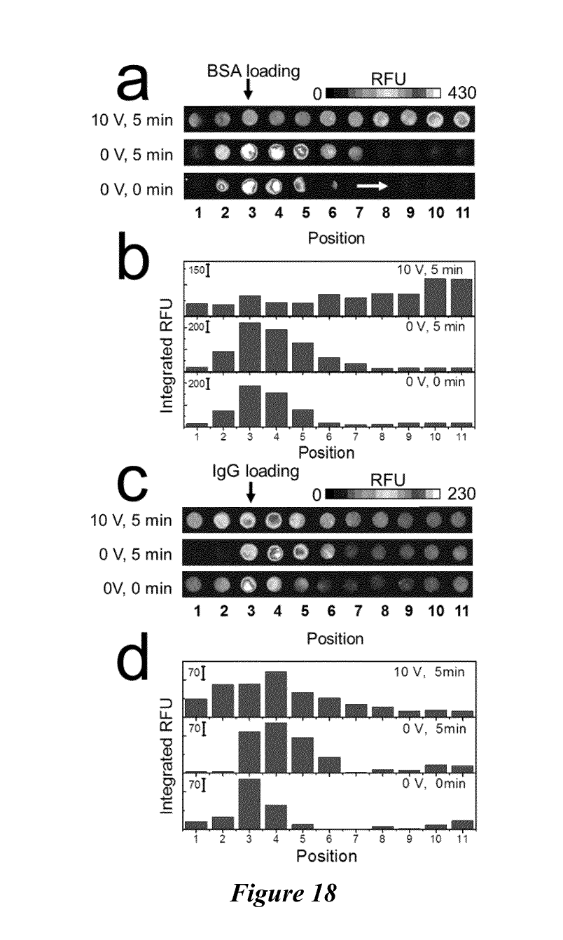

FIG. 18a shows fluorescence micrographs of BSA in the oPAD-Ep before and after the application of 10.0 V for 5.0 min, and after 5.0 min in the absence of an electric field. When no voltage was applied, BSA underwent random diffusion, spreading out by ˜1 layer from the initial position within 5.0 min. In contrast, when 10.0 V was applied, BSA migrated towards the cathode at a speed of ˜1 layer/min (or 3 μm/s). The electric field was estimated as 10.0 V divided by the thickness of 10-layer origami paper (˜1.8 mm), giving a value of ˜5.5 kV/m. Equation 2 was used to calculate the mobility of BSA, which was found to be ˜5×10⁻¹⁰ m²/(s·V).

The mobility of BSA measured in the oPAD-Ep is an order of magnitude lower than the value reported in the literature using conventional paper Ep (Kunkel H G and Tiselius A. J. Gen. Physiol. 1951, 35, 89-118). In the previously reported experiments, however, Ep was carried out for 14 h (150 times longer than in these experiments) to achieve a reasonable separation of serum proteins. This long immersion time can cause deterioration of the paper structure, which can lead to faster migration of BSA. This contention is supported by the small difference (<8%) between the measured mobility of BSA in paper and in free solution noted in this prior report (Kunkel H G and Tiselius A. J. Gen. Physiol. 1951, 35, 89-118). In addition, the type of paper and the pH used is different, and the effects of electroosmosis were not considered in the calculations. After migration, remnants of BSA were observed on the paper from the oPAD-Ep (Positions 6-9, FIGS. 18a and b), which can be attributed to nonspecific adsorption of BSA in paper based devices (Scida K et al. Anal. Chem. 2013, 85, 9713-9720).

In contrast to BSA, the distribution of IgG shifted only slightly toward the anode after 5.0 min (FIGS. 18c and d). This can be because IgG has a different isoelectric point than BSA: 7.3±1.0 (Josephson R et al. J. Dairy Sci. 1972, 55, 1508-1510) and 4.9±0.1 (Abramson H A et al. Electrophoresis of Proteins and the Chemistry of Cell Surfaces; Hafner Publishing Company, Inc.: New York, 1942; Dawson R M C. Data for Biochemical Research, 3rd; Clarendon Press: Oxford, 1989), respectively (recall that the separation was carried out at pH 7.4). Additionally, IgG is a larger molecule (˜150 kDa) than BSA (˜66.5 kDa) (Putnam F W, Ed. The Plasma Proteins V3: Structure, Function, and Genetic Control; Vol. 3. Elsevier: London, 2012), which can also lead to a lower mobility.

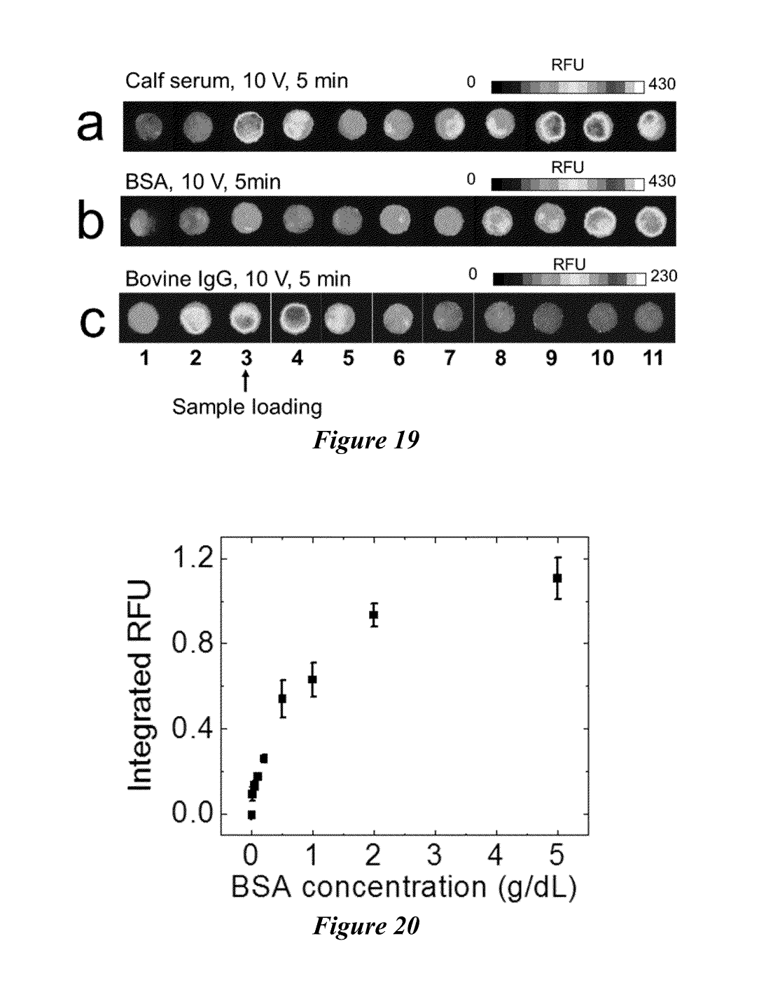

Applying the same conditions used for the control experiments illustrated in FIG. 18 (e.g., BSA and IgG as separate solutions), a separation of the components of calf bovine serum was carried out (e.g., a mixture containing both BSA and IgG). FIG. 19a is a fluorescence micrograph of an oPAD-Ep after separation of a 0.50 μL calf serum sample for 5.0 min at 10.0 V. Two fluorescence maxima are apparent: one near the starting location of the separation (Position 3), which belongs to immunoglobulin proteins (including IgG), and the other near Positions 9-10, corresponding to BSA. By simple visual comparison with the fluorescence intensities of the control experiments (FIG. 18, and reproduced in FIGS. 19b and c for ease of comparison), it is possible to obtain a quick semi-quantitative analysis. The total amount of BSA in the calf serum (FIG. 19a) was close to that of the BSA control of 5.0 g/dL (FIG. 19b), which lies in the normal range of 3-5 g/dL (Kyle R et al. Clinical Indications and Applications of Electrophoresis and Immunofixation. In Manual of Clinical Immunology; Rose N et al., Eds.; ASM Press: Washington D.C. 2002; pp 66-70). Comparison of FIGS. 19a and c revealed that the immunoglobulin protein concentration was higher than 1.0 g/dL IgG, but still in a reasonable range considering that immunoglobulin proteins other than IgG are also present in the calf serum sample.

Paper zones with a diameter of 3.5 mm were used to obtain a BSA calibration curve. These paper zones were first wetted with 0.50 μL 0.1×PBS solution (ionic strength 16.3 mM), and then 0.50 μL of BSA solutions having different concentrations were spotted at the wet paper zone, followed by another 0.50 μL epicocconone to stain the protein. After that, the devices were kept in a humidity chamber for 30.0 min to allow epicocconone to fully react with BSA, and then moved to a dark room until the samples were dry (˜30 min). After taking fluorescence micrographs, the RFU of each paper zone was integrated in ImageJ and plotted as a function of BSA concentration (FIG. 20). The calibration curve shows that the fluorescence intensity of protein starts to deviate from linearity at concentrations >˜0.50 g/dL. As the concentrations of BSA and IgG in the calf serum used in FIG. 19 were above 0.5 g/dL, a more quantitative analysis of the results shown in FIG. 19 was not possible. Also, the Alpha-1, Alpha-2 and Beta bands, which usually appear between the immunoglobulin proteins and albumin in conventional serum Ep, could not be distinguished by the oPAD-Ep (FIG. 19). This can be because of the strong background of non-specifically absorbed BSA.

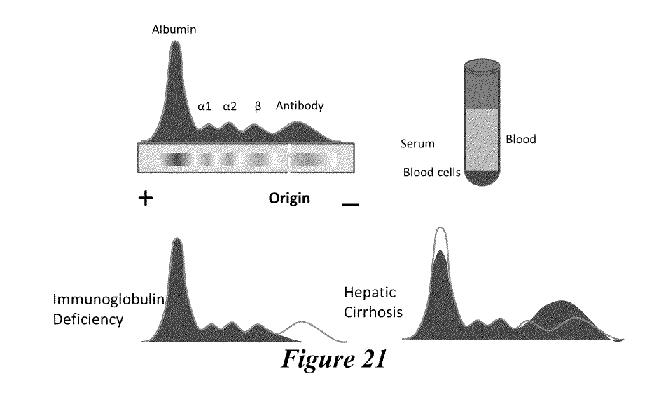

A low-cost separation system based on folded paper has been described. This approach takes advantage of the thinness of origami paper (180 μm/layer) to achieve a high electric field strength (several kV/m) at a low applied voltage (˜10 V). The voltage required for the oPAD-Ep is more than an order of magnitude lower than that used in conventional electrophoretic devices. The simple construction, low voltage requirement, and ease of use can make the oPAD-Ep a candidate for POC applications (e.g., for separation of serum proteins as illustrated in FIG. 21). Moreover, because it is able to separate fluorescent molecules and serum proteins within ˜5 min, it can be integrated into other types of paper-based devices for pre-separation of samples, for example blood components.

### Example 2

In this example, use of devices for isotachophoretic (ITP) preconcentration of charged molecules, such as DNA is described.

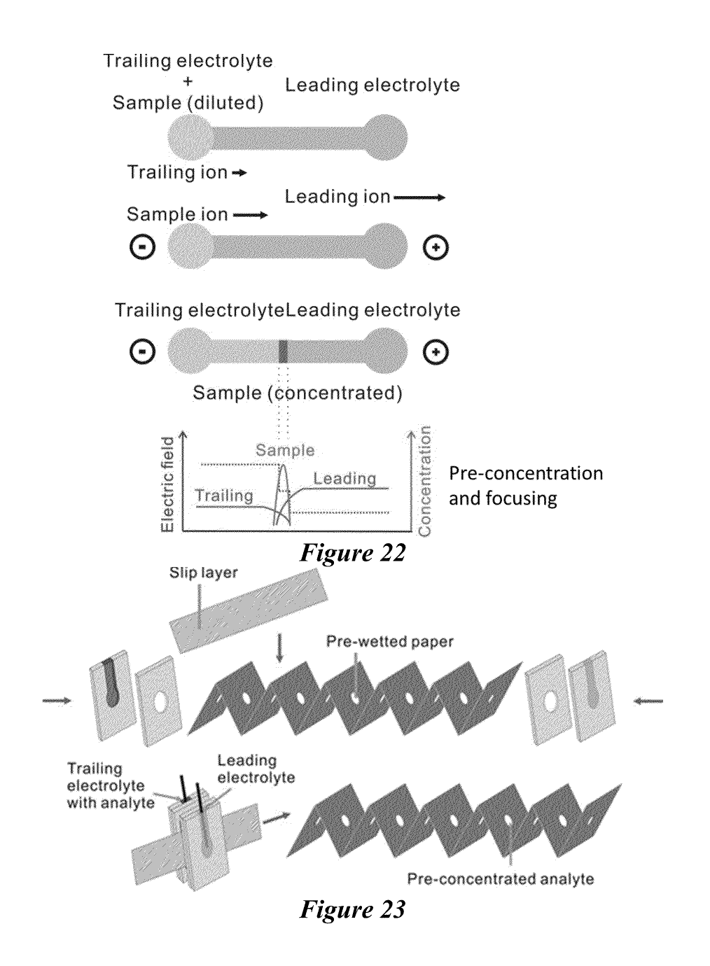

Isotachophoresis (ITP) is a special form of electrophoresis (Ep), and can be used for selective separation and preconcentration of analytes with specific ionic mobilities (e.g., DNA). ITP can use different electrolyte solutions in the cathode and anode buffer reservoirs: one with a higher ionic mobility, referred to as the leading electrolyte (LE), and the other one with a lower ionic mobility, referred to as the trailing electrolyte (TE) (FIG. 22). When an analyte has a mobility in between the LE and TE, it can be collected and focused as a thin band (˜100 μm in the case of microfluidic ITP) between the two electrolytes in an electric field. Otherwise, the analyte is not collected and focused. Similar functionality can be achieved, but with a much lower voltage, using the devices described herein (˜a few hundred volts in microfluidic ITP vs. 10-20 V in the device described herein).

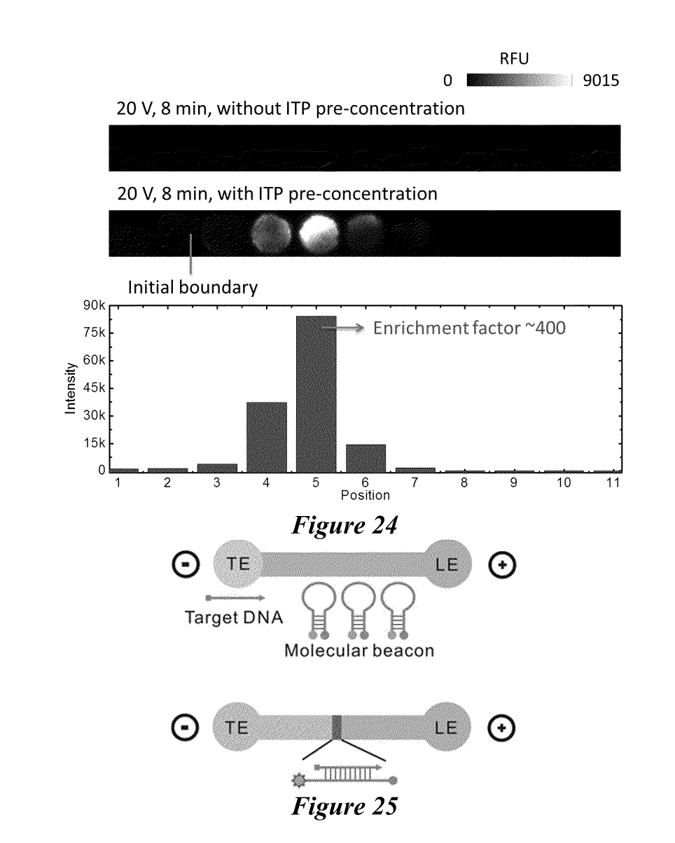

The ITP method using the devices described herein is illustrated generally in FIG. 23. The method can comprise filling the two buffer reservoirs with LE and TE, respectively. A Slip layer B can be used to separate the LE and TE and establish an initial sharp boundary between them. After applying a voltage bias between the two electrodes, removing the slip layer can initiate ITP, and any low-concentration DNA in the TE can be accumulate and focused at the LE/TE boundary. The results shown in FIG. 24 show that 1) >50% of the total DNA in the TE was collected and focused on 1-2 layers of the multilayer paper at 20 V for 8 min; 2) the DNA concentration was increased by ˜400 fold as a result of this process. The paper layer with concentrated DNA can be cut out and used for further analyses.

Preconcentration of specific targets before analysis by the method discussed herein can lower the limit of detection (LOD) on a paper device, at least by 2 to 3 orders of magnitude (e.g., FIG. 25). In addition, the device and method describe herein can be used for selective separation of biomolecules from cell lysate based on their mobility difference, such as obtaining DNA or RNA from cell lysate. The simple construction, low voltage requirement, and ease of use can make the device and method described herein a candidate for POC applications, such as for diagnosing Hepatitis B, Hepatitis C, AIDS, etc. using DNA or RNA from cell lysate.

### Example 3

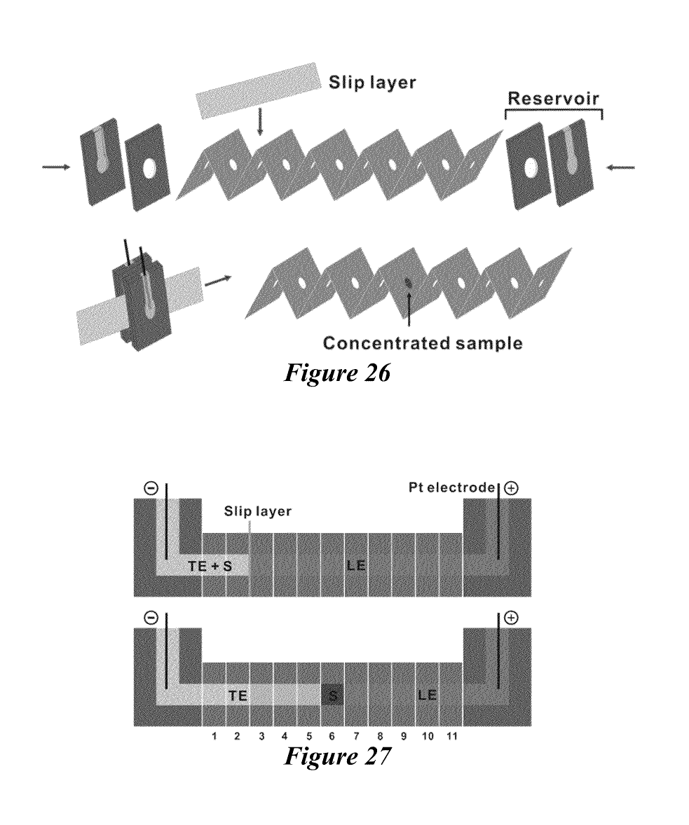

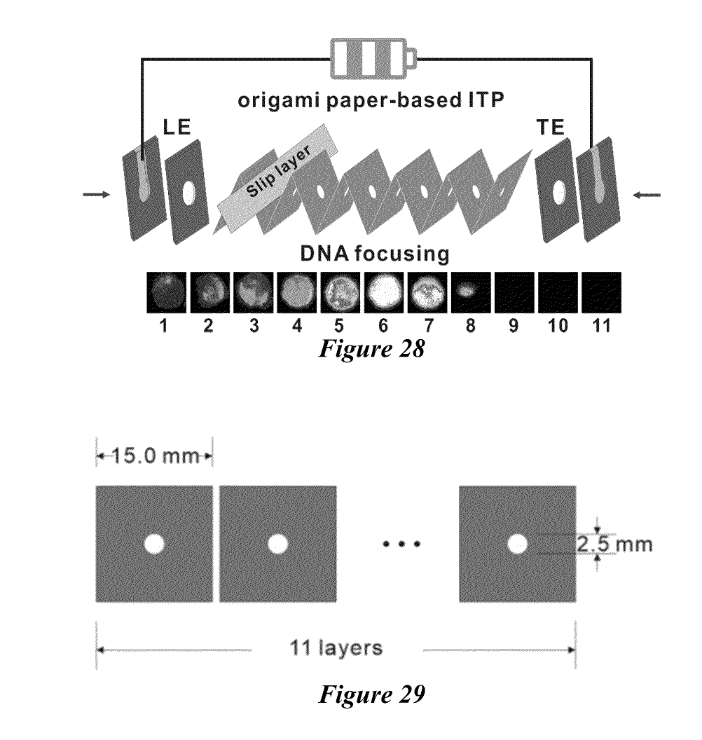

Herein, a paper isotachophoresis (ITP) platform fabricated using the principles of origami (Japanese paper folding) is described (Jung B et al. Anal. Chem., 2006, 78, 2319-2327). The device can be inexpensive, easy to assemble and operate, and can electrokinetically concentrate DNA. The design of this origami paper analytical device (oPAD) for isotachophoresis (oPAD-ITP) is illustrated in FIG. 26 and FIG. 27 (Liu H and Crooks R M. J. Am. Chem. Soc., 2011, 133, 17564-17566; Li X et al. Lab Chip. 2015, 15, 4090-4098). Briefly, a piece of wax-patterned paper (Carrilho E et al. Anal. Chem., 2009, 81, 7091-7095) is folded into a concertina configuration, a plastic slip layer (Du W B et al. Lab Chip, 2009, 9, 2286-2292; Liu H et al. Anal. Chem., 2013, 85, 4263-4267; Shen F et al. Lab Chip, 2010, 10, 2666-2672) is inserted into one of the folds, and then this assembly is sandwiched between reservoirs for the trailing electrolyte (TE) and leading electrolyte (LE). DNA, present at concentrations on the order of 10⁻⁹ M, is initially mixed with the trailing electrolyte solution and then added to the trailing electrolyte reservoir, followed by addition of the leading electrolyte solution to its reservoir. These solutions flow spontaneously into the paper channel but are prevented from mixing by the slip layer. Next, a voltage bias is applied between electrodes in the reservoirs, and then the slip layer is removed. This results in accumulation of DNA (e.g., 100-fold concentration amplification) at the interface of the two electrolytes, for example, within ˜4 min. A general schematic of DNA focusing using the oPAD-ITP is shown in FIG. 28.

The power requirements of the oPAD-ITP for the examples described herein were supplied by two 9 V batteries. This is a >20-fold lower voltage than previously reported, and therefore true point-of-care (POC) applications can be accessible and complications due to Joule heating can be minimized (Rosenfeld T and Bercovici M. Lab Chip, 2014, 14, 4465-4474; Moghadam B Y et al. Anal. Chem., 2014, 86, 5829-5837; Moghadam B Y et al. Anal. Chem., 2015, 87, 1009-1017). Further, the origami paper channel is fully enclosed by wax, and therefore solvent (e.g., water) evaporation can be minimized. The plastic slip layer can be used to establish a well-defined initial trailing electrolyte/leading electrolyte boundary. The oPAD-ITP is “digital” in the sense that the enriched product will be on individual paper layers and can be reclaimed by simply cutting off the desired layer(s). This opens up the possibility of coupling the oPAD-ITP with other detection systems to achieve lower limits of detection (LOD).

Due to their biocompatibility (Martinez A W et al. Anal. Chem., 2010, 82, 3-10), ease of fabrication (Carrilho E et al. Anal. Chem., 2009, 81, 7091-7095), and low-cost (Martinez A W et al. Anal. Chem., 2010, 82, 3-10; Carrilho E et al. Anal. Chem., 2009, 81, 5990-5998), PADs can be used as POC diagnostic devices and systems (Gubala V et al. Anal. Chem., 2011, 84, 487-515). A number of detection methods have been developed for PADs, including: electrochemistry (Scida K et al. Anal. Chem., 2013, 85, 9713-9720; Scida K et al. Anal. Chem., 2014, 86, 6501-6507; Dungchai W et al. Anal. Chem., 2009, 81, 5821-5826), photography (Ellerbee A K et al. Anal. Chem., 2009, 81, 8447-8452; Zhao W et al. Anal. Chem., 2008, 80, 8431-8437), luminescence (Ge L et al. Biomaterials, 2012, 33, 1024-1031; Ge L et al. Chem. Commun., 2014, 50, 5699-5702; Zhang X et al. Chem. Commun., 2013, 49, 3866-3868; Delaney J L et al. Anal. Chem., 2011, 83, 1300-1306), and others (Parolo C and Merkoci A. Chem. Soc. Rev., 2013, 42, 450-457; Yetisen A K et al. Lab Chip, 2013, 13, 2210-2251). Nevertheless, it still can be difficult to achieve sufficiently low limits of detection for some important applications, particularly those involving nucleic acid detection. One way around this problem is sample preconcentration, and, although there are many ways to approach this for bulk solutions, the number that have been reported for paper platforms is limited (Yetisen A K et al. Lab Chip, 2013, 13, 2210-2251).

Isotachophoresis (ITP) is an electrophoretic technique that can effectively concentrate ionic samples with minimal sample pretreatment (Jung B et al. Anal. Chem., 2006, 78, 2319-2327; Walker P A et al. Anal. Chem., 1998, 70, 3766-3769; Persat A et al. Anal. Chem., 2009, 81, 9507-9511). In a typical isotachophoresis experiment, the electric field profile across an electrophoretic channel is controlled by using electrolytes having different mobilities: a fast moving leading electrolyte and a slow moving trailing electrolyte (Everaerts F M et al. Isotachophoresis: theory, instrumentation and applications, Elsevier, 2011). When a voltage is applied across the channel, sample ions, initially present in the trailing electrolyte solution, out-pace the trailing electrolyte and accumulate at the trailing electrolyte/leading electrolyte boundary, which can result in a high local concentration.

Herein, an oPAD design for electrophoretic separations was adapted for isotachophoresis, such as for isotachophoresis concentration of DNA. Four experiments are described velow in more detail. First, isotachophoresis of 23-mer single-stranded DNA labeled with Cyanine5 (ssDNA) using the oPAD-ITP is discussed and this result is compared to a mathematical model (Rosenfeld T and Bercovici M. Lab Chip, 2014, 14, 4465-4474). Second, the effect of the initial concentration of sample DNA on isotachophoresis enrichment is examined. Third, some fundamental principles of the oPAD-ITP are examined, such as the electric field profile during sample focusing. Finally, an application of the oPAD-ITP for the isotachophoresis of a 100 bp dsDNA ladder, which is comprised of 100-1517 bp of double-stranded DNA (dsDNA), is discussed. The results of these studies suggest that the oPAD-ITP can provide additional functionality for a variety of paper-based detection platforms.

Whatman Grade 1 cellulose paper, HCl, acetic acid, and agarose were purchased from Fisher Scientific (Walthman, Mass.). Single-stranded DNA (ssDNA, 5′-AGT CAG TGT GGA AAA TCT CTA GC-Cy5-3′) was ordered from Integrated DNA Technologies (Coralville, Iowa) and purified by HPLC. The 100 bp dsDNA ladder was from New England BioLabs (Ipswich, Mass.). The following chemicals were from Sigma-Aldrich (St. Louis, Mo.) and used as received: 2-amino-2-(hydroxymethyl)-1,3-propanediol (tris base), 2-aminoethanesulfonic acid (taurine), Ru(bpy)₃Cl₂, ethidium bromide (EtBr) solution (10 mg/mL), and EDTA.

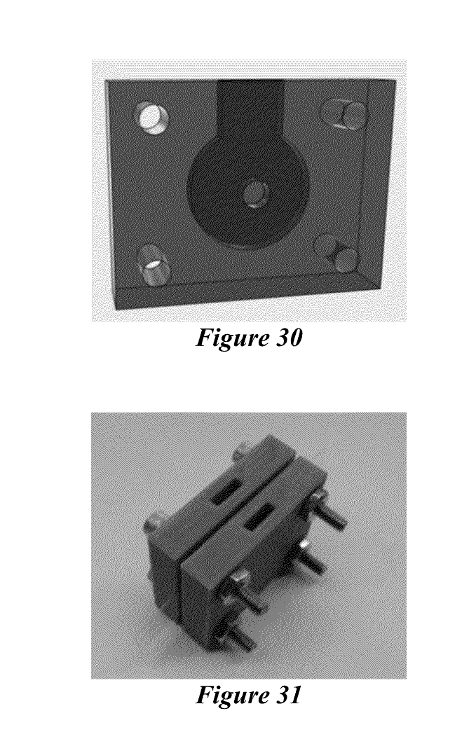

The fabrication of the oPAD-ITP is similar to that of an electrophoretic device reported previously (Luo L et al. Anal. Chem., 2014, 86, 12390-12397), but there are some important differences. Whatman grade 1 cellulose paper (˜180 μm thick) was patterned with wax (CorelDRAW designs shown in FIG. 29) using a Xerox ColorQube 8750DN inkjet printer. A ˜2 mm-diameter wax-free region is present in the center of each section of the device in the pattern. The patterned paper was placed in an oven at 120° C. for 45 s to allow the wax to penetrate through the thickness of paper (Carrilho E et al. Anal. Chem., 2009, 81, 7091-7095), and then cooled to 25±2° C. After folding the paper into an 11-layer origami structure (FIG. 26), a piece of plastic sheet (photo laminating sheets from 3M: 0.5 mm thick, 4.0 cm long, and 1.0 cm in wide) was inserted between the second and third layers. This slip layer is used to form the initial trailing electrolyte/leading electrolyte boundary and also serves as a switch to initiate the isotachophoresis process. The trailing electrolyte and leading electrolyte reservoirs were fabricated from acrylonitrile butadiene styrene (FIG. 30) using a Flashforge Pro XL 3D printer. The assembled oPAD-ITP was then sandwiched between the two reservoirs. The degree of compression of the concertina fold was controlled using four screws situated at the corners of the reservoirs (FIG. 31).

The trailing electrolyte and leading electrolyte solutions used in these experiments were 2.0 mM tris-taurine (pH 8.7) and 1.0 M tris-HCl (pH 7.3), respectively. After assembling the oPAD-ITP (origami paper, slip layer and reservoirs), the reservoirs were filled with 1.0 mL of the trailing electrolyte or leading electrolyte buffer as shown in FIGS. 26-28. The circular paper channel was completely wetted within 1 min. A Pt wire was inserted into each reservoir, and then two 9 V batteries were connected in series and used as the power supply (18 V in total).

For the ssDNA concentration experiments, 40.0 nM of the ssDNA was initially present in the trailing electrolyte solution. For the dsDNA ladder experiments, 0.50 μg/mL of the dsDNA ladder was initially mixed in the trailing electrolyte solution. In the electric field profiling experiment, 30.0 μM Ru(bpy)₃²⁺ was initially mixed in the leading electrolyte solution. For these latter experiments, Ag/AgCl electrodes (Lathrop D K et al. J. Am. Chem. Soc., 2010, 132, 1878-1885), rather than Pt, were used to avoid generation of Cl₂ due to the low resistance and high current level (˜17 mA). After isotachophoresis experiments, the solutions in both reservoirs were removed and the device was disassembled to analyze the content of each paper layer.

A Nikon AZ100 multi-purpose zoom fluorescence microscope with Nikon filters (ssDNA: 590-650 nm excitation and 663-738 nm emission; Ru(bpy)₃²⁺: 420-490 nm excitation and 510-700 nm emission) was used to acquire fluorescence images of each fold of the oPAD-ITP device. All images were then processed with ImageJ software to obtain integrated relative fluorescence unit (RFU) intensity for quantification of fluorescent molecules on each layer.

Gel electrophoresis was used to quantify the dsDNA content on each paper layer after isotachophoresis of the dsDNA ladder. Gel electrophoresis was chosen for two reasons. First, most common dsDNA stains, such as SYBR gold or EtBr, exhibit a high background on cellulose paper, and this can make it difficult to visualize and quantify the amount of dsDNA. Second, the dsDNA ladder is comprised of twelve dsDNA components having lengths ranging from 100 to 1517 bp. Gel electrophoresis can separate them and provides quantitative information for each component of the ladder.

The gel electrophoresis analyses were conducted as follows. First, each fold of the paper was cut off, dried, and then inserted into a 1.3% agarose gel containing 10 μg/mL EtBr (FIG. 32). Control samples were prepared by drying 1.0 μL of the 500 μg/mL dsDNA ladder stock solution in the paper zone. Gel electrophoresis was run using 1×TAE (containing 40.0 mM tris, 20.0 mM acetic acid, and 1.0 mM EDTA) buffer for 50 min at 100 V (Lambda LLS9120 DC Power Supply). A Typhoon Trio fluorescence scanner (GE Healthcare, Piscataway, N.J.) was used to image the gel, followed by ImageJ software analysis.

The ssDNA focusing experiments were carried out as follows. An 11-layer oPAD-ITP was assembled as shown in FIGS. 26-28, with the insulating slip layer initially placed between the second and third paper folds. Next, two 3D-printed reservoirs were filled with 1.0 mL of 2.0 mM tris-taurine (trailing electrolyte) containing 40.0 nM ssDNA and 1.0 M tris-HCl (leading electrolyte), respectively. Electrodes were placed into the reservoirs, an 18 V bias was applied, and the slip layer was removed.

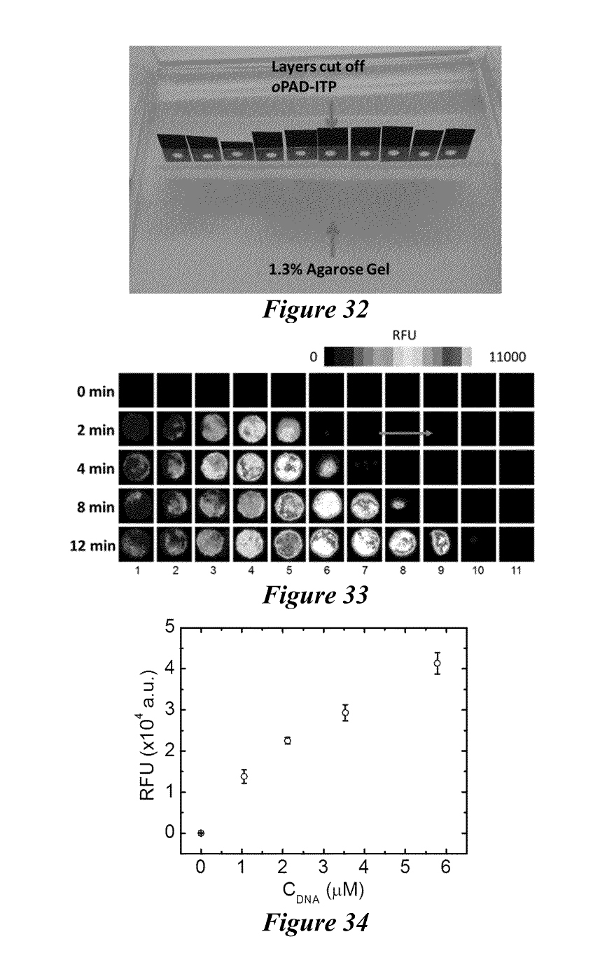

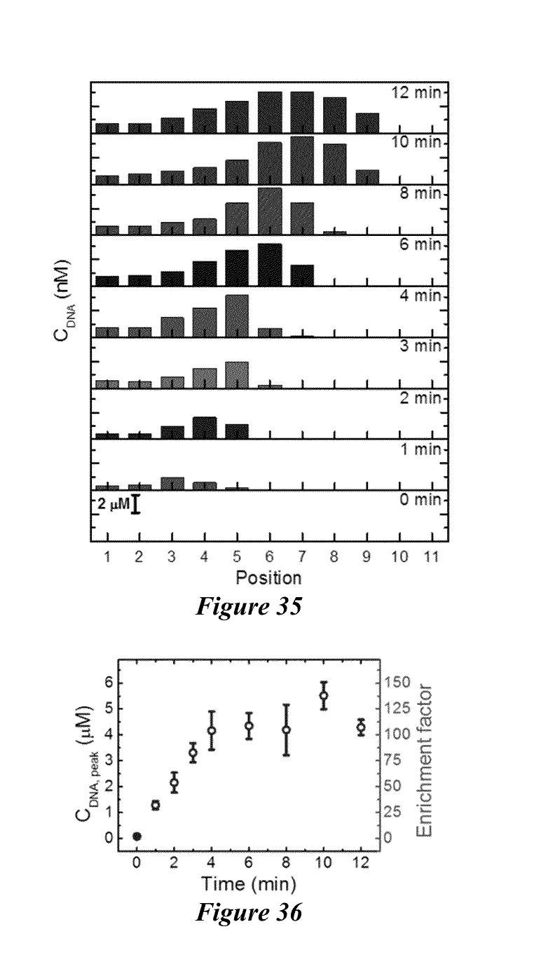

FIG. 33 shows fluorescence micrographs of each paper layer as a function of time during isotachophoresis focusing of ssDNA. The fluorescence intensities increase with time, indicating accumulation of ssDNA from the buffer. The majority of the concentrated ssDNA is distributed between 2 to 4 paper layers, which correspond to a width of ˜0.4-0.7 mm (the average thickness of a single layer of paper is ˜180 μm) (Carrilho E et al. Anal. Chem., 2009, 81, 7091-7095). The precise concentration of ssDNA in each layer was determined by integrating the fluorescence intensity and then comparing it to a standard calibration curve (FIG. 34). Typical concentration histograms of ssDNA as a function of position and time are shown in FIG. 35. FIG. 36 shows that the peak (maximum) concentration of ssDNA (C_{DNA,peak}) grows linearly at a rate of ˜1 μM/min until it reaches a plateau of ˜4 μM at ˜4 min. This corresponds to a ˜100-fold enrichment of ssDNA from the initial 40.0 nM concentration. This enrichment factor is averaged over a thickness of 180 μm, and so it does not represent a true peak concentration. In other words, the oPAD-ITP can be considered a digital device, with each paper fold representing one “bin.”

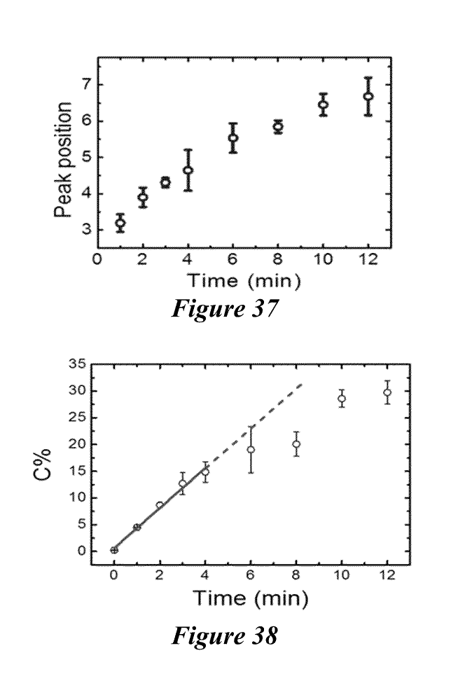

In microfluidic isotachophoresis experiments, sample ions are focused at the trailing electrolyte/leading electrolyte boundary, where there is a sharp change in the magnitude of the electric field. As the experiment progresses, this boundary migrates toward the leading electrolyte reservoir. FIG. 33 shows that this behavior was qualitatively replicated in the oPAD-ITP. That is, the location of maximum ssDNA concentration migrates from left to right, mirroring the location of the trailing electrolyte/leading electrolyte boundary. This result is quantified in FIG. 37. Here, the peak positions were determined by Gaussian fitting of the ssDNA distributions (FIG. 35), and then plotted as a function of time. This relationship is nearly linear, and the slope of this plot (˜0.3 layers/min) represents the velocity of the trailing electrolyte/leading electrolyte boundary. The mobility of the trailing electrolyte/leading electrolyte boundary (μ_{ITP}) can be estimated using equation 3.

$${μ}_{I T P} = \frac{a \cdot d}{E} \qquad ( 3 )$$

Here, a is the slope of the linear fit in FIG. 37, d is the thickness of one paper layer (˜180 μm), and E is the electric field strength. E can be estimated by dividing the applied voltage (18 V) by the total thickness of the 11-layer origami paper. This assumes that the majority of the potential drop occurs in the paper channel rather than the reservoirs, which, given the higher resistance of the paper, is reasonable. The calculated value of μ_{ITP} is 1.08×10⁻¹⁰ m²/s·V, which is one order of magnitude smaller than its counterpart in conventional microfluidic channels or other paper isotachophoresis devices (˜10⁻⁹ m²/s·V) (Jung B et al. Anal. Chem., 2006, 78, 2319-2327; Rosenfeld T and Bercovici M. Lab Chip, 2014, 14, 4465-4474). A possible explanation is that all ions are forced to travel through the three-dimensional cellulose matrix in the oPAD-ITP. This can increase the true migrational distance (tortuous path), and, in addition, can result in specific interactions between the ions and the cellulose fibers. This view is consistent with previously reported findings that the mobilities of ions through paper are about one order of magnitude smaller than in free solution (Luo L et al. Anal. Chem., 2014, 86, 12390-12397). In other paper-based isotachophoresis devices, ions migrate laterally across the paper (rather than normal to it, which is the case for the oPAD-ITP), which can lead to different migrational pathways (Rosenfeld T and Bercovici M. Lab Chip, 2014, 14, 4465-4474; Moghadam B Y et al. Anal. Chem., 2014, 86, 5829-5837).

In isotachophoresis, the collection efficiency (C %) is defined as the percentage of the original sample that is accumulated by an isotachophoresis device during a defined period of time. The collection efficiency (C %) for the oPAD-ITP was calculated using equation 4 (Persat A et al. Anal. Chem., 2009, 81, 9507-9511).

$$C \% = \frac{{\sum }_{j = 1}^{11} {C}_{j} {V}_{j}}{{C}_{0} {V}_{T E}} \qquad ( 4 )$$

Here, Cⱼ is the concentration of ssDNA on the jₜₕ layer and Vⱼ is the liquid capacity of one paper layer, ˜0.5 μL. Co and V_{TE} are the original sample concentration and the volume of the trailing electrolyte solution, respectively. The calculated value of the collection efficiency (C %) is plotted as a function of time in FIG. 38. Between 0 and 4 min, the collection efficiency increases linearly with time at a rate of ˜4%/min. At longer times, however, the collection efficiency increases at a lower rate. The maximum value of the collection efficiency is ˜30%, which is obtained after 12 min. The slower accumulation rate at long times can be related to the lowering of the ssDNA concentration in the reservoir as the experiment progresses. The broadened distribution of the focused ssDNA after 4 min (FIGS. 33 and 35) can affect the ion concentration profile near the trailing electrolyte/leading electrolyte boundary, which can disrupt the local electric field.

In isotachophoresis, the extraction efficiency is the ability of the device to concentrate a defined sample volume per unit electrical charge consumed. A descriptor, η, can be used represent the extraction efficiency and can be calculated using equation 5 (Rosenfeld T and Bercovici M. Lab Chip, 2014, 14, 4465-4474). A high value of η means less energy is required to concentrate a sample.

$$η = \frac{{N}_{D N A } ( t )}{{C}_{0} {\int }_{0}^{t} i ( t ) d t} \qquad ( 5 )$$

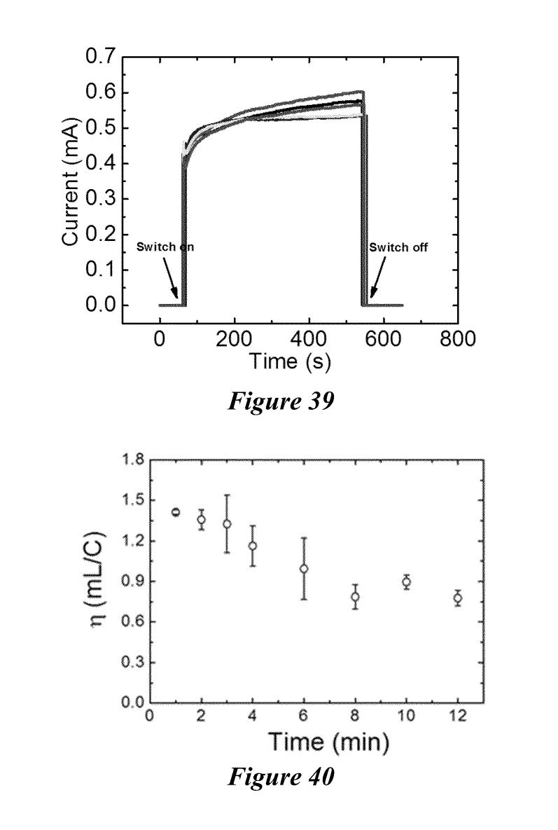

Here, N_{DNA}(t) is the total moles of ssDNA focused by the oPAD-ITP after t min. In the experiments discussed herein, the current, i(t), remained almost constant at ˜0.53 mA during the focusing process (FIG. 39), and N_{DNA}(t) is linearly correlated with time for the first 4 min of the experiment (FIG. 38). Therefore, η is 0.9-1.5 mL/C (FIG. 40), which is 3-5 times higher than the value reported by Bercovici (0.3 mL/C) (Rosenfeld T and Bercovici M. Lab Chip, 2014, 14, 4465-4474). The higher η value observed herein experiments can be caused by the lower trailing electrolyte concentration (2.0 mM taurine/2.0 mM tris) used for operation of the oPAD-ITP, compared to that used by Rosenfeld and Bercovici (10 mM tricine/20 mM bistris). When lower concentrations of trailing electrolyte are used, the sample ions can carry a higher percentage of the total current, and this can lead to better extraction efficiencies.

According to classical peak-mode isotachophoresis theory, when the sample concentration is negligible compared with the concentration of either electrolyte (trailing electrolyte and leading electrolyte), the maximum peak sample concentration (C_{sample,peak}) depends solely on the trailing electrolyte and leading electrolyte composition, and is independent of the initial sample concentration (CO) (Jung B et al. Anal. Chem., 2006, 78, 2319-2327). Therefore, the enrichment factor (EF), defined as the value of C_{sample,peak} divided by Co, will be inversely proportional to the value of CO. Accordingly, the oPAD-ITP performance was examined using different initial ssDNA concentrations (C_{DNA,0}), but otherwise the same experimental procedure described above.

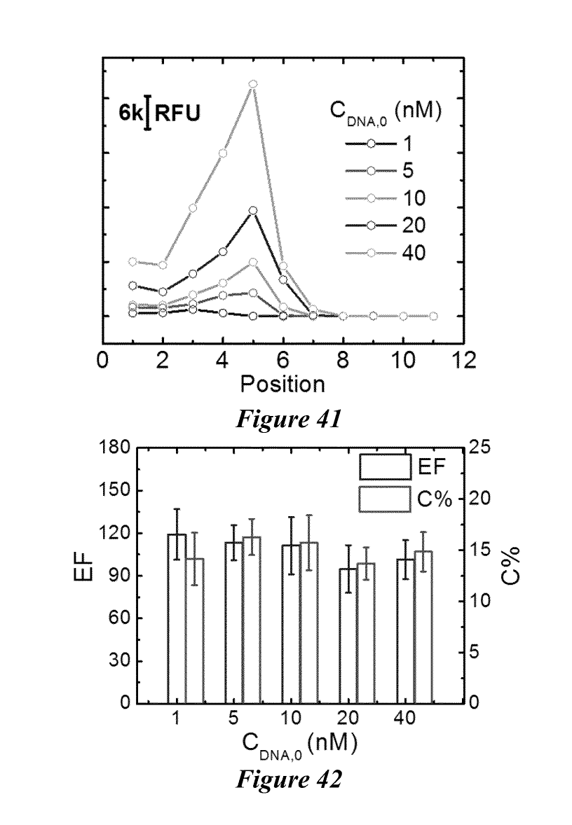

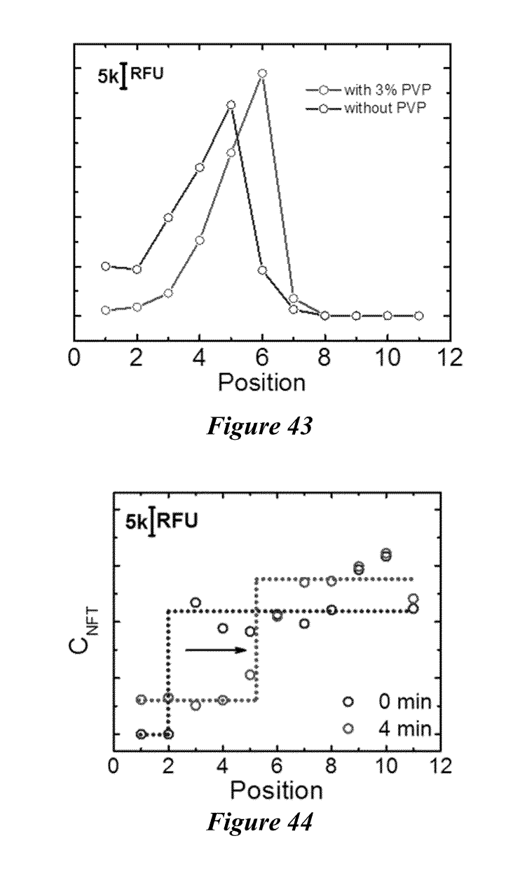

FIG. 41 shows the distribution of accumulated ssDNA as a function of its position within the oPAD-ITP and initial ssDNA concentration (C_{DNA,0}) after 4 min of isotachophoresis focusing. The concentration profiles shown in this figure are similar in shape, indicating that the electric field in the devices is not a strong function of initial ssDNA concentration. Enrichment factors (EFs) and collection efficiencies (C % s) as a function of initial ssDNA concentration (C_{DNA,0}) are presented in FIG. 42. Both quantities have roughly constant values of ˜100 and 15%, respectively, as initial ssDNA concentration varies from 1.0 nM to 40.0 nM. This finding is in contrast to the expectation that both quantities should be inversely related to initial ssDNA concentration. There are several possible explanations for this observation. First, the accumulation process during the first 4 min could have been limited by the migration of ssDNA within the cellulose matrix and did not reach the theoretical maximum accumulation. Therefore, ssDNA accumulates in the channel at a constant rate regardless of the initial concentration (C_{DNA,0}), leading to constant enrichment factor and collection efficiency values. Second, as mentioned before, the value of enrichment factor is calculated from the peak ssDNA concentration (C_{DNA,peak}), which is averaged over the thickness of a 180 μm paper fold. This can introduce some uncertainty into the determination of enrichment factors. Third, electroosmotic flow (EOF) can play a role in isotachophoresis focusing by generating a counter flow in the paper channel. This can slow ssDNA migration and broaden the peak. However, a control experiment shown in FIG. 43 does not support this idea, because the enrichment factor is unchanged in the presence and absence of electroosmotic flow.

In microfluidic devices, focusing of analyte at the trailing electrolyte/leading electrolyte boundary can result from a sharp transition of the electric field between the trailing electrolyte and leading electrolyte (Chambers R D and Santiago J G. Anal. Chem., 2009, 81, 3022-3028). To investigate if the same is true for the oPAD-ITP, the electric field profile during isotachophoresis focusing was measured, according to a previously reported approach (Chambers R D and Santiago J G. Anal. Chem., 2009, 81, 3022-3028). Specifically, Ru(bpy)₃²⁺, a nonfocusing fluorescent tracer (NFT) (Chambers R D and Santiago J G. Anal. Chem., 2009, 81, 3022-3028), was added to the leading electrolyte solution, and then its distribution across the paper folds in the oPAD-ITP was determined after focusing. In principle, the nonfocusing fluorescent tracer will migrate through the channel during isotachophoresis and leave behind a concentration distribution that is inversely proportional to the local electric field strength.

FIG. 44 shows the distribution of the nonfocusing fluorescent tracer concentration (C_{NFT}) in an oPAD-ITP at t=0 and 4 min. These data were obtained using the same experimental conditions used for the ssDNA focusing experiments. At t=0 min, the nonfocusing fluorescent tracer is only present in the leading electrolyte zone (Layers 3 to 11), and there is a concentration step between Layer 2 and 3. This step represents the trailing electrolyte/leading electrolyte boundary where the slip layer was initially located. At t=4 min, the concentration step is still present, but it has moved three positions to the right. This is the same location where ssDNA accumulated after 4 min of isotachophoresis (FIG. 33), thereby further indicating that ssDNA accumulation occurs at the trailing electrolyte/leading electrolyte boundary. Based on the value of the nonfocusing fluorescent tracer concentration in the channel, the electric field strength in the leading electrolyte and trailing electrolyte zones can be approximated as 3 and 16 kV/m, respectively.

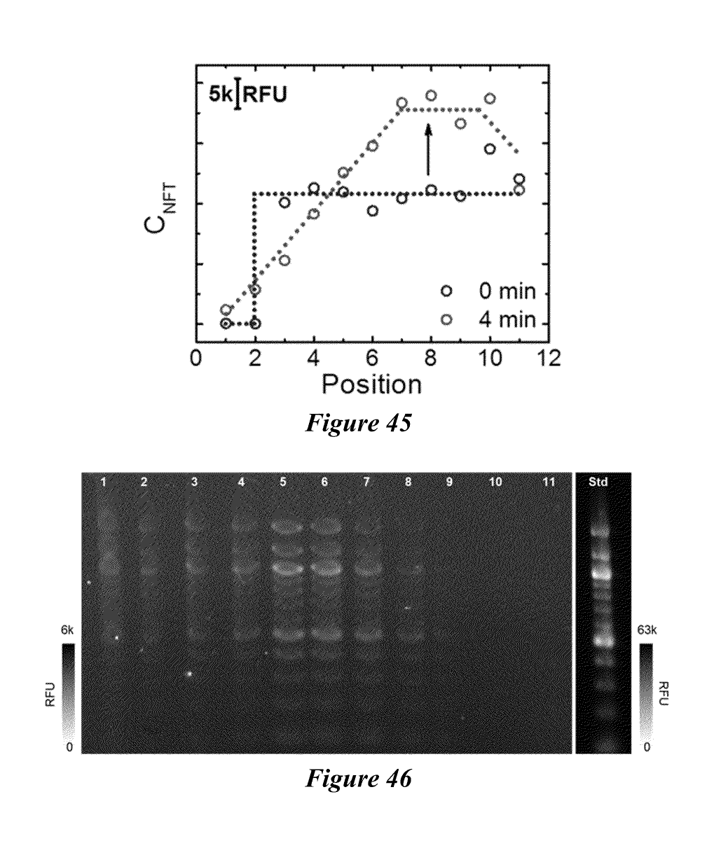

There is a possibility that the step-shaped distribution of nonfocusing fluorescent tracer shown in FIG. 44 (t=4 min) could arise from slow migration of nonfocusing fluorescent tracer in the paper matrix. Accordingly, a control experiment was performed to examine this possibility. Instead of using two different electrolytes (trailing electrolyte and leading electrolyte), the same electrolyte (1.0 M tris-HCl) was loaded into both reservoirs, though the nonfocusing fluorescent tracer is only placed in the leading electrolyte reservoir. This condition should result in a uniform electric field across the entire channel, and therefore the nonfocusing fluorescent tracer concentration step should disappear after applying the voltage, if the step indeed represents the trailing electrolyte/leading electrolyte boundary and not slow migration of nonfocusing fluorescent tracer in the paper matrix. FIG. 45 shows the result of this experiment. At t=0 min, a step-shaped concentration profile is observed, just as in FIG. 44. After 4 min, however, the nonfocusing fluorescent tracer concentration step is replaced by a trapezoidal distribution: a linear increase from Layer 1 to 7, a plateau from Layer 7 to 10, and finally a decrease at Layer 11. This distribution can be caused by the mobility differences of the nonfocusing fluorescent tracer in the paper medium and in free solution (Luo L et al. Anal. Chem., 2014, 86, 12390-12397), which determines the value of the nonfocusing fluorescent tracer concentration near the paper/reservoir boundary (boundary effect). As shown in FIG. 45, accumulation of nonfocusing fluorescent tracer is observed near the right paper/reservoir boundary, due to larger influx of nonfocusing fluorescent tracer from that reservoir into the paper. In the contrast, depletion of nonfocusing fluorescent tracer near the left paper/reservoir boundary results from larger out-flow of nonfocusing fluorescent tracer from paper into that reservoir.

Based on the results in this section, ssDNA focusing can be caused by the electric field transition at the trailing electrolyte/leading electrolyte boundary, and, to a lesser extent, by boundary effect.

Even though short DNA strands (usually several tens of bases) are widely used as model targets for developing DNA sensing technologies, real-world DNA, for example, in viruses or bacteria, is usually composed of thousands of base pairs (Lodish H F et al. Molecular cell biology, Citeseer, 2000; Davis L. Basic methods in molecular biology, Elsevier, 2012). Accordingly, it can be desirable for an isotachophoresis device to be capable of focusing DNA strands longer 100 bp. In this section, the oPAD-ITPs were used for focusing a 100 bp dsDNA ladder containing 100-1517 bp dsDNA. The same experimental setup and buffer conditions used in the previous section were used for these experiments. That is, the dsDNA ladder was loaded into the trailing electrolyte buffer, and the voltage was switched on for 10 min.

After isotachophoresis, each fold of paper was removed from the channel, and gel electrophoresis was used to elute its dsDNA content (FIG. 32). The dsDNA on the gel was stained by EtBr and then imaged using a fluorescence scanner. A raw fluorescence image of a typical gel is shown in FIG. 46. Here, each lane represents one paper layer. The right-most lane was used as a standard (the dsDNA ladder solution was dropcast onto a single paper fold, but it was not exposed to isotachophoresis). The results in FIG. 46 indicate that the concentrations for the different dsDNA lengths achieved their maximum values at Layers 5 and 6. Recall that the peak position for the shorter ssDNA was at Layer 7 (FIG. 35). The reduced mobility of the longer dsDNA ladder can be due to its stronger affinity for the cellulose matrix (Araújo A C et al. Anal. Chem., 2012, 84, 3311-3317).

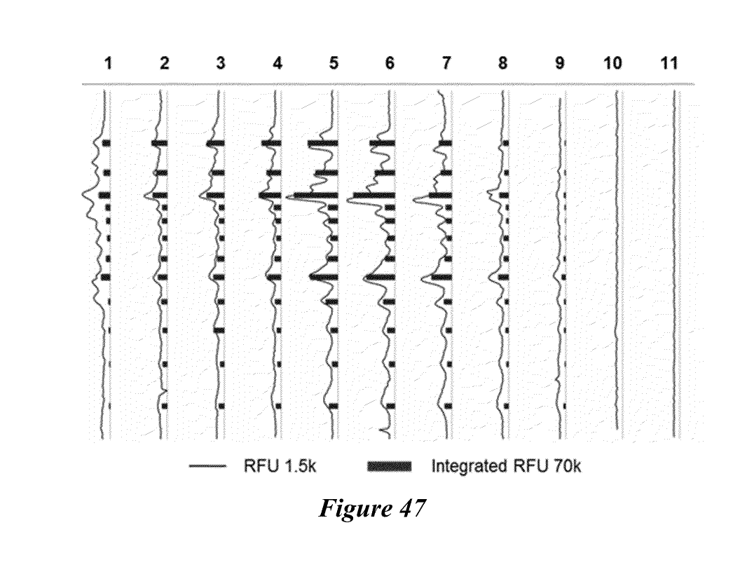

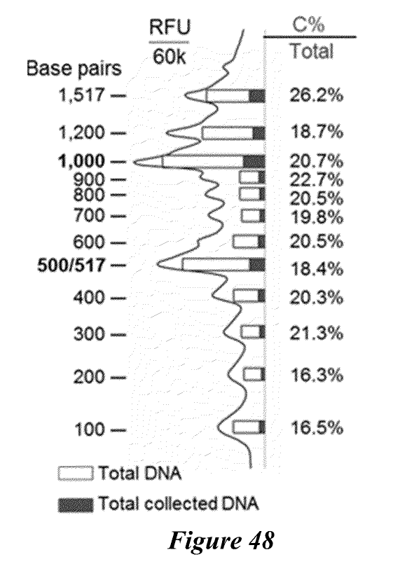

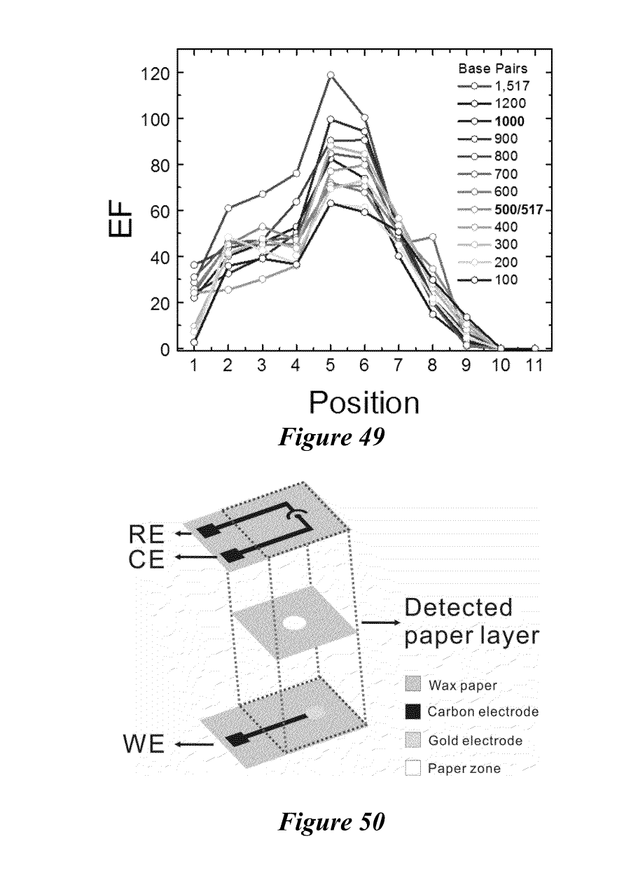

FIG. 47 presents line profiles (solid lines) of the relative fluorescence unit (RFU) intensity corresponding to the dsDNA bands in FIG. 46. The integrated relative fluorescence unit (RFU) values of each dsDNA band, representing the dsDNA amount, are shown as solid bars aligned with the line profiles. FIG. 48 is the same analysis for the ladder standard (right side of FIG. 46): the lines present the relative fluorescence unit (RFU) line profiles, the hollow bars show the integrated relative fluorescence unit value of each dsDNA band, and the solid bars equal the sum of the dsDNA amount on individual paper folds (equivalent to the solid bars in FIG. 47). The enrichment factor for each dsDNA length was calculated as the area of solid bars in FIG. 47 divided by the area of the hollow bars in FIG. 48. FIG. 49 is a plot of the enrichment factor as a function of the layer number for different dsDNA lengths. The maximum enrichment factors are found at Layer 5 and they vary from 60 to 120, which is consistent with the ssDNA results. The collection efficiency values of each dsDNA length were obtained as the area of the solid bars divided by the area of the hollow bars in FIG. 48. The right column of FIG. 48 shows that the collection efficiency for all dsDNA lengths ranges from ˜15-20%. These results indicate that focusing of dsDNA having different lengths (up to 1517 bp) in the oPAD-ITP yields a consistent collection efficiency of ˜20% and enrichment factor of ˜100.

Herein, an origami paper-based device suitable for carrying out low-voltage isotachophoresis, the oPAD-ITP, was used for focusing of DNA samples having lengths ranging from 23 to at least 1517 bp. DNA was concentrated by more than two orders of magnitude within 4 min. The device uses a 2 mm-long, 2 mm-wide circular paper channel formed by concertina folding a paper strip and aligning the circular paper zones on each layer. Due to the short channel length, a high electric field of ˜16 kV/m can be generated in the paper channel using two 9 V batteries. The multiplayer architecture can also enable reclamation and analysis of the sample after isotachophoresis focusing by opening the origami paper and cutting out the desired layers. The electric field in the origami paper channel during isotachophoresis experiments was profiled using a nonfocusing fluorescent tracer. The result showed that focusing can rely on formation and subsequent movement of an electric field boundary between the leading and trailing electrolyte.

This approach can resolves several issues of previously reported paper-based isotachophoresis designs, including high operating voltage, solvent evaporation, and difficult sample reclamation. Using the oPAD-ITP, a >100-fold enrichment of ssDNA and dsDNA having lengths of up to 1517 bps was demonstrated. The time required for enrichment was ˜10 min, the paper device can accommodate solution volumes of up to 1.0 mL, and is battery operated (18 V). The collection efficiency ranged from ˜15-20%. The electric field profiling experiments, using Ru(bpy)₃²⁺ as a tracer, indicated that the focusing mechanism in the oPAD-ITP can be the same as in bulk liquid solutions: accumulation of sample at the boundary between the trailing electrolyte and leading electrolyte.

The oPAD-ITP can be coupled with other paper-based detection system to achieve lower limits of detection (Scida K et al. Anal. Chem., 2014, 86, 6501-6507). The structure of the paper channels can be tailored to achieve better sample enrichment.

### Example 4

In the examples above, fluorescence has been used to analyze analytes on each paper layer of an origami device. Here, electrochemical methods were used for quantitative analysis of the analytes on a paper layer. As shown in FIG. 50, a paper layer containing analytes in a hydrophilic zone at the center can serve as an electrochemical cell. This cell can be sandwiched between two layers of wax paper containing patterned electrodes (e.g., a working electrode (WE) on one layer of wax paper and a reference electrode (RE) and counter electrode (CE) on the second layer of wax paper). By applying an appropriate potential wave function at the electrodes, the analyte on the paper layer can be oxidized or reduced. Quantitative information about the analyte can then be obtained from the electrochemical current signal. This technique can provide for electrochemical detection on a single paper layer, and can be integrated into oPAD-Ep systems to provide for separation, enrichment, and detection on a single paper layer. The detection of silver nanoparticles (AgNPs), a common probe (electrochemical tag) for bio-sensing, on a single paper layer is described below to illustrate this principle.

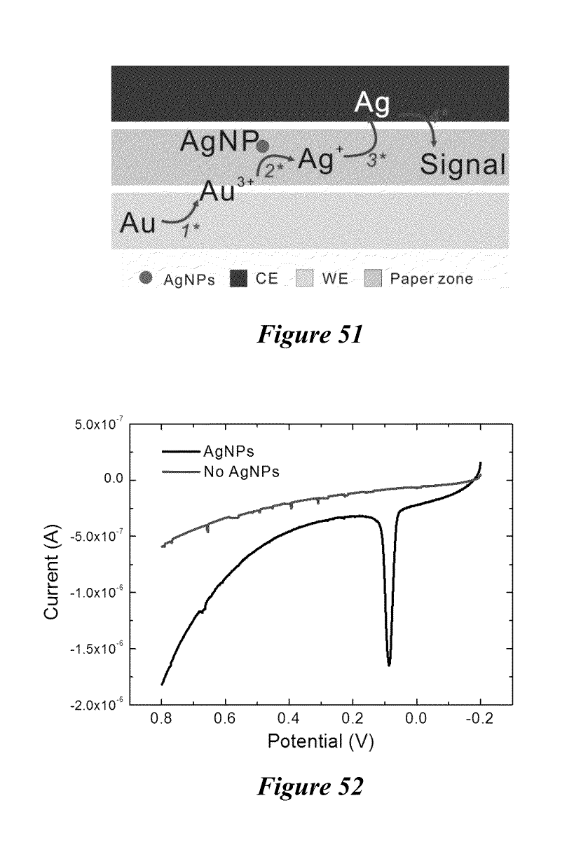

FIG. 51 illustrates a scheme for the electrochemical detection of AgNPs. The paper layer in which the electrochemical detection is performed (with AgNPs in PBS buffer) is sandwiched between two paper layers to form a three-electrode system. All three electrodes are screen printed carbon electrodes and the working electrode (WE) is pre-modified with gold (Au). First, a positive potential is applied at WE to oxidize Au to generate Au³⁺ (Step 1*). Once formed, Au³⁺ undergoes a galvanic exchange reaction with the AgNPs to generate Ag⁺ (Step 2*). Meanwhile, due to the opposite negative potential on the counter electrode (CE), the Ag⁺ is electro-deposited on the surface of CE (Step 3*). Next, linear stripping voltammetry (LSV) is performed on the CE by changing the potential of CE from positive to negative to re-oxidize the Ag on the CE surface (Step 4*). The total charge of Ag peak in the voltammogram corresponds to the AgNPs amount on the detected paper layer, allowing for quantification of the AgNPs (and by extension an analyte conjugated to (labeled with) the AgNP.

FIG. 52 shows the linear stripping voltammogram obtained as a result. A PBS solution containing 565 pM AgNPs was detected in the paper layer. The positive signal is shown in black trace. The negative control, gray trace, was obtained using the same procedure, except in the absence of AgNPs in the initial PBS solution.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

What is claimed is:

1. A device comprising:
a stack formed from a plurality of parallel segments;
a fluid permeable column traversing the stack from a first end to a second end;
a first electrode in electrical contact with the first end; and
a second electrode in electrical contact with the second end;
wherein each segment comprises a fluid permeable region defined by a fluid impermeable boundary; and
wherein stacking of the plurality of segments aligns the fluid permeable region within each of the plurality of parallel segments to form the fluid permeable column.

2. The device of claim 1, further comprising a slip layer, wherein the slip layer comprises:
a fluid permeable region defined by a fluid impermeable boundary;
wherein the slip layer can be translocated from a retracted position to a deployed position;
wherein in the retracted position the fluid permeable region of the slip layer is fluidly independent from the fluid permeable column; and
wherein in the deployed position, the fluid permeable region of the slip layer is in fluid contact with the fluid permeable column.

3. The device of claim 1, wherein the plurality of segments are joined together in a sheet, and the stack is formed by folding the sheet.

4. The device of claim 3, wherein folding the sheet comprises accordion folding the sheet.

5. The device of claim 1, wherein the plurality of parallel segments comprises at least 3 parallel segments.

6. The device of claim 1, wherein the fluid permeable column is 10 mm or less in length.

7. The device of claim 1, wherein the device is paper based.

8. A method comprising:
introducing a sample to the fluid permeable column of the device of claim 1; and
applying a potential to the fluid permeable column.

9. The method of claim 8, wherein the sample comprises an analyte, and wherein the method further comprises separating the analyte from the sample.

10. The method of claim 9, further comprising accumulating the sample, the analyte, or a combination thereof in a section of the fluid permeable column.

11. The method of claim 10, further comprising removing the section of the fluid permeable column to isolate the sample, the analyte, or a combination thereof.

12. The method of claim 10, wherein the section can comprise one or more of the parallel segments, a slip layer, or a combination thereof.

13. The method of claim 10, further comprising analyzing the sample, analyte, or a combination thereof to determine a property of the sample, the analyte, or a combination thereof.

14. The method of claim 13, wherein analyzing the sample, analyte, or a combination thereof comprises fluorescence spectroscopy of the sample, analyte, or a combination thereof.

15. The method of claim 13, wherein analyzing the sample, analyte, or a combination thereof comprises electrochemical analysis of the sample, analyte, or a combination thereof.

16. The method of claim 8, wherein the device further comprising a slip layer, wherein the slip layer comprises:
a fluid permeable region defined by a fluid impermeable boundary;
wherein the slip layer can be translocated from a retracted position to a deployed position;
wherein in the retracted position the fluid permeable region of the slip layer is fluidly independent from the fluid permeable column; and
wherein in the deployed position, the fluid permeable region of the slip layer is in fluid contact with the fluid permeable column; and
wherein introducing the sample to the fluid permeable column comprises translocating the slip layer to the deployed position, wherein the sample is initially located in the fluid permeable region of the slip layer.

17. A device comprising:
a plurality of planar segments, each planar segment comprising:
a top surface;
a bottom surface; and
a fluid permeable region defined by a fluid impermeable boundary extending through the planar segment from the top surface to the bottom surface so as to form a fluid permeable pathway extending through the planar segment from the top surface to the bottom surface;
wherein when the plurality of planar segments are stacked such that the bottom surface of a first planar segment is in intimate contact with the top surface of a second planar segment, the fluid permeable regions together form a fluid permeable column within the stacked plurality of planar segments extending from a first end to a second end;
wherein the first end comprises the fluid permeable region at the top surface of the first planar segment;
wherein the second end with the fluid permeable region at the bottom surface of the last planar segment;
a first electrode in electrical contact with the first end;
a second electrode in electrical contact with the second end.

18. The device of claim 17, further comprising a slip layer, wherein the slip layer comprises a fluid permeable region defined by a fluid impermeable boundary, wherein the slip layer can be translocated from a retracted position to a deployed position, wherein in the retracted position the fluid permeable region of the slip layer is fluidly independent from the fluid permeable column, and wherein in the deployed position, the fluid permeable region of the slip layer is in fluid contact with the fluid permeable column.

19. The device of claim 17, wherein the device is paper based, wherein the plurality of planar segments comprise at least 3 planar segments joined together in a sheet, and wherein the plurality of planar segments are stacked to form a fluid permeable column is 10 mm or less in length by accordion folding the sheet.

20. A device comprising:
a stack formed from a plurality of parallel segments;
a first fluid permeable column traversing the stack from a first end to a second end;
a second fluid permeable column traversing the stack from a third end to a fourth end;
a first electrode in electrical contact with the first end, the third end, or a combination thereof; and
a second electrode in electrical contact with the second end, the fourth end, or a combination thereof;
wherein each segment comprises a first fluid permeable region defined by a first fluid impermeable boundary and a second fluid permeable region defined by a second fluid impermeable boundary; and
wherein stacking of the plurality of segments aligns the first fluid permeable region within each of the plurality of parallel segments to form the first fluid permeable column and the second fluid permeable region within each of the plurality of parallel segments to form the second fluid permeable column.


SEQUENCE LISTING (1 sequence)

SEQ ID NO: 1 - DNA, 24 bp, Artificial Sequence
  synthetic construct
agtcagtgtggaaaatctctagcn